SEQUENCE LISTING (4 sequences)

SEQ ID NO: 1 - RNA, 44 nt, synthetic construct
  source 1..44: mol_type = other RNA
gggggnnnannannnnannnagngnnanganaannnncccanca

SEQ ID NO: 2 - RNA, 35 nt, synthetic construct
  source 1..35: mol_type = other RNA
gggggnnnannannnnannnagngnnanganaann

SEQ ID NO: 3 - RNA, 30 nt, synthetic construct
  source 1..30: mol_type = other RNA
gggnnannannnnannnagngnnanganaa

SEQ ID NO: 4 - RNA, 23 nt, synthetic construct
  source 1..23: mol_type = other RNA
gnggnaggaagaannggaagngn

(19) United States
(12) Patent Application Publication
(10) Pub. No.: US 2025/0383350 A1
(43) Pub. Date: Dec. 18, 2025

(54) ANALYTE DETECTION USING APTAMERS

(71) Applicants: The Regents of the University of Michigan, Ann Arbor, MI (US); ALIGHT SCIENCES, INC., Ann Arbor, MI (US)

(72) Inventors: Tanmay Chatterjee, Ann Arbor, MI (US); Alexander Johnson-Buck, Ann Arbor, MI (US); Nils Walter, Ann Arbor, MI (US)

(21) Appl. No.: 18/876,226

(22) PCT Filed: Jul. 13, 2023
(86) PCT No.: PCT/US2023/070159

§ 371 Date: Dec. 18, 2024

Related U.S. Application Data
(60) Provisional application No. 63/389,406, filed on Jul. 15, 2022.

(51) Int. Cl. G01N 33/563; G01N 33/53; G01N 33/543; G01N 33/557; G01N 33/58; G01N 33/68

(52) U.S. Cl. CPC G01N 33/563; G01N 33/5308; G01N 33/543; G01N 33/557; G01N 33/582; G01N 33/6803

(57) ABSTRACT

Provided herein is technology relating to detecting analytes and particularly, but not exclusively, to methods, compositions, systems, and kits for detecting analytes using aptamer technologies.

This application claims priority to U.S. provisional patent application Ser. No. 63/389,406, filed Jul. 15, 2022, which is incorporated herein by reference in its entirety.

### PARTIES OF JOINT RESEARCH AGREEMENT

The present disclosure was made by or on behalf of the below listed parties to a joint research agreement. The joint research agreement was in effect on or before the date the present disclosure was made, and the present disclosure was made as a result of activities undertaken within the scope of the joint research agreement. The parties to the joint research agreement are: aLight Sciences, Inc. and the Regents of the University of Michigan.

### FIELD

Provided herein is technology relating to detecting analytes and particularly, but not exclusively, to methods, compositions, systems, and kits for detecting analytes using aptamer technologies.

### BACKGROUND

Sensitive and accurate detection, quantification, identification, and/or characterization of biomarkers finds use in clinical diagnostics for differentiating between healthy and diseased states. Accordingly, diagnosis and treatment of disease would benefit from new technologies for rapid and accurate analysis of biomarkers.

### SUMMARY

Single Molecule Recognition through Equilibrium Poisson Sampling (SiMREPS) has emerged as a powerful technique for the ultrasensitive and specific detection of protein and other biomarkers, with LODs in the aM to low fM range (see, e.g., U.S. Pat. No. 10,093,967; U.S. Pat. App. Pub. Nos. 2021/0348230; 2021/0230688; 2021/0292837; 2018/0258469; 2019/0187031; 2021/0318296; and U.S. Pat. App. Ser. No. 63/224,984, each of which is incorporated herein by reference). The high sensitivity of SiMREPS results from use of binding and dissociation kinetics to distinguish signals of specific binding of query probes to an analyte from nonspecific binding (e.g., to assay surfaces or matrix) or binding to non target analytes. In particular, previous SiMREPS technologies have used antibody query probes with relatively fast dissociation kinetics (k_{off} of approximately 0.05-0.5 s⁻¹). The antibody query probes repeatedly associate and dissociate with target analytes, which provides a repeated interrogation of single target molecules and generates characteristic kinetic fingerprints within a reasonably short acquisition time (e.g., 2 min per field of view) without sacrificing sensitivity.

In some embodiments, provided herein is a technology comprising use of aptamers as detection probes in SiMREPS assays. Aptamers are synthetic single-stranded DNA (ssDNA) or RNA oligonucleotides that fold into unique three-dimensional structures and bind their targets specifically. Use of aptamers in SiMREPS detection provides several advantages. First, generating aptamers by systematic evolution of ligand by exponential enrichment (e.g., systematic evolution of ligands by exponential enrichment (SELEX); see, e.g., Gold (2015), “SELEX: How It Happened and Where It will Go” Journal of Molecular Evolution 81 (5-6): 140-143; and Ellington (1990) “In vitro selection of RNA molecules that bind specific ligands” Nature 346 (6287): 818-22, each of which is incorporated herein by reference) and related methods (e.g., cyclic amplification and selection of targets (CAST) or selected and amplified binding site (SAAB); see, e.g., Wright (1991) “Cyclic amplification and selection of targets (CASTing) for the myogenin consensus binding site” Molecular and Cellular Biology 11 (8): 4104-10; and Blackwell (1990) “Differences and similarities in DNA binding preferences of MyoD and E2A protein complexes revealed by binding site selection” Science 250 (4984): 1104-10, each of which is incorporated herein by reference) can be performed quickly and at low cost, e.g., more quickly and at lower cost than generating an antibody against a target protein by hybridoma or phage display technology. Moreover, aptamers can be synthesized by solid-phase chemical synthesis, which permits aptamers to be synthesized more easily and more reproducibly than antibodies. Further, in some embodiments, aptamers comprised modified bases (e.g., modified with 2′-fluoro or 2′-O—CH₃ groups) to increase their stability and resistance to nuclease degradation. Other advantages of aptamers include their non toxicity and non-immunogenicity, reduced steric hindrance due to their smaller sizes, ease of modification, and thermal stability.

Experiments conducted during the development of the technology described herein used aptamers as detection probes in SiMREPS assays. In particular, experiments were conducted to use aptamer query probes to detect two clinically relevant protein biomarkers: VEGF₁₆₅ and IL-8. Samples comprising spiked-in VEGF₁₆₅ and endogenous human IL-8 in serum matrices were assayed using a wash-free SiMREPS detection protocol. Data collected from these experiments indicated limits of detection in the low femtomolar range (e.g., 3.1 fM or 0.026 pg/mL for IL-8 detection; and 8.9 fM or 0.340 pg/mL for VEGF₁₆₅ detection).

Furthermore, data collected from these experiments indicated that aptamers can be rationally optimized for use as dynamically binding SiMREPS query probes by incorporating small sequence modifications into the aptamers during chemical synthesis. Exemplary sequence modifications that were tested in the experiments included providing one or more nucleotide substitutions in a conserved region or shortening adjacent helical stems. The experiments indicated that aptamer sequences can be modified to provide association dissociation kinetics that are useful for SiMREPS methods and that provide an improved rate of data acquisition and better distinction between signal and background kinetic fingerprints. Data indicated that chemical synthesis of aptamers provides for the site-specific and stoichiometric labelling of aptamers at sites that do not affect analyte binding. Thus, design and synthesis of aptamers having a desired binding affinity for an analyte are simplified because the effects of site-specific labeling on binding affinity are minimized and/or eliminated. In addition, using stoichiometrically labelled aptamers as SiMREPS query probes provides a two-state intensity signal that directly characterizes the kinetics of query probe association-dissociation without noise or signal complexity due to multiply nonstoichiometric labelling.

Accordingly, in some embodiments, the technology provides a method for detecting an analyte. For example, in some embodiments, the method comprises stably binding an analyte to a solid support; providing an analyte specific aptamer query probe comprising a detectable label; and recording a time-dependent change in a signal intensity of the detectable label. In some embodiments, the solid support comprises an immobilized capture probe and stably binding the analyte to the solid support comprises stably binding the analyte to the immobilized capture probe. In some embodiments, the detectable label comprise a fluorescent moiety. In some embodiments, the solid support is diffusible.

In some embodiments, the analyte comprises a protein. In some embodiments, the analyte comprises a nucleic acid. In some embodiments, the analyte comprises a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism.

In some embodiments, the analyte is present at a concentration of 1 to 10 fM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 fM). In some embodiments, the analyte is present at a concentration of 0.01 to 1 pg/mL (e.g., 0.01, 0.02, 0.05, 0.1, 0.2, 0.5, or 1 pg/mL).

In some embodiments, the capture probe comprises an antibody or antigen-binding antibody fragment. In some embodiments, the capture probe comprises a nucleic acid. In some embodiments, transient association of the query probe with the analyte produces the time-dependent change in the signal intensity of the detectable label.

In some embodiments, the method further comprises counting a number of changes in the signal intensity of the detectable label. In some embodiments, the method further comprises determining a value for N_{b+d}. In some embodiments, the method further comprises determining a value for τ_{on, median}. In some embodiments, the method further comprises providing a sample comprising the analyte. In some embodiments, the sample is a biological sample. In some embodiments, stably binding the analyte to the solid support comprises contacting the sample to the solid support. In some embodiments, the method further comprises identifying a candidate aptamer and introducing a number of single-nucleotide changes into the conserved target-binding region of the candidate aptamer to produce the aptamer query probe. In some embodiments, identifying the candidate aptamer comprises using in vitro evolution (e.g., SELEX). In some embodiments, the method further comprises truncating the candidate aptamer.

In some embodiments, recording the time dependent change in the signal intensity of the detectable label comprises recording a series of images. In some embodiments, the method further comprises producing an intensity fluctuation map by determining an average absolute image-to-image change in intensity at a number of image pixels. In some embodiments, the method further comprises generating intensity-versus time data and calculating a kinetic parameter from the intensity-versus-time data. In some embodiments, the method further comprises identifying positive detection events using a threshold for the kinetic parameter.

In some embodiments, the technology provides a system for detecting an analyte (e.g., using a SiMREPS assay, e.g., as described in U.S. Pat. No. 10,093,967; U.S. Pat. App. Pub. Nos. 2021/0348230; 2021/0230688; 2021/0292837; 2018/0258469; 2019/0187031; 2021/0318296; and U.S. Pat. App. Ser. No. 63/224,984, each of which is incorporated herein by reference). In some embodiments, the system comprises a solid support; an analyte specific aptamer query probe comprising a detectable label; a detector configured to detect the detectable label; a memory configured to record time-dependent changes in a signal intensity of the detectable label; and a processor configured to generate intensity-versus-time data from the time-dependent changes in a signal intensity of the detectable label.

In some embodiments, the system further comprises an analyte. In some embodiments, the analyte is stably bound to the solid support. In some embodiments, the solid support comprises a capture probe. In some embodiments, the detectable label comprise a fluorescent moiety. In some embodiments, the solid support is diffusible.

In some embodiments, the analyte comprises a protein. In some embodiments, the analyte comprises a nucleic acid. In some embodiments, the analyte comprises a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism. In some embodiments, the analyte is present at a concentration of 1 to 10 fM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 fM). In some embodiments, the analyte is present at a concentration of 0.01 to 1 pg/mL (e.g., 0.01, 0.02, 0.05, 0.1, 0.2, 0.5, or 1 pg/mL).

In some embodiments, the capture probe comprises an antibody or antigen-binding antibody fragment. In some embodiments, the capture probe comprises a nucleic acid.

In some embodiments, transient association of the query probe with the analyte produces the time-dependent change in the signal intensity of the detectable label.

In some embodiments, the processor is further configured to count a number of changes in the signal intensity of the detectable label. In some embodiments, the processor is further configured to determine a value for N_{b+d}. In some embodiments, the processor is further configured to determine a value for τ_{on, median}. In some embodiments, the processor is configured to record a series of images. In some embodiments, the processor is configured to produce an intensity fluctuation map by determining an average absolute image-to-image change in intensity at a number of image pixels. In some embodiments, the processor is configured to calculate a kinetic parameter from the intensity versus-time data. In some embodiments, the processor is configured to identify positive detection events using a threshold for the kinetic parameter.

Further, the technology provided herein is related to use of an analyte-specific aptamer query probe to characterize, identify, quantify, and/or detect an analyte in a SiMREPS assay method (e.g., as described in U.S. Pat. No. 10,093,967; U.S. Pat. App. Pub. Nos. 2021/0348230; 2021/0230688; 2021/0292837; 2018/0258469; 2019/0187031; 2021/0318296; and U.S. Pat. App. Ser. No. 63/224,984, each of which is incorporated herein by reference). In some embodiments, the SiMREPS assay method comprises: stably binding the analyte to a solid support; providing the analyte-specific aptamer query probe comprising a detectable label; and recording a time-dependent change in a signal intensity of the detectable label. In some embodiments, the solid support comprises an immobilized capture probe and stably binding the analyte to the solid support comprises stably binding the analyte to the immobilized capture probe. In some embodiments, the detectable label comprise a fluorescent moiety. In some embodiments, the solid support is diffusible.

In some embodiments, the analyte comprises a protein. In some embodiments, the analyte comprises a nucleic acid. In some embodiments, the analyte comprises a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism. In some embodiments, the analyte is present at a concentration of 1 to 10 fM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 fM). In some embodiments, the analyte is present at a concentration of 0.01 to 1 pg/mL (e.g., 0.01, 0.02, 0.05, 0.1, 0.2, 0.5, or 1 pg/mL).

In some embodiments, the capture probe comprises an antibody or antigen-binding antibody fragment. In some embodiments, the capture probe comprises a nucleic acid.

In some embodiments, transient association of the query probe with the analyte produces the time-dependent change in the signal intensity of the detectable label.

In some embodiments, the SiMREPS assay method further comprises counting a number of changes in the signal intensity of the detectable label.

In some embodiments, the SiMREPS assay method further comprises determining a value for N_{b+d}. In some embodiments, the SiMREPS assay method further comprises determining a value for τ_{on, median}.

In some embodiments, the use comprises providing a sample comprising the analyte. In some embodiments, the sample is a biological sample. In some embodiments, stably binding the analyte to the solid support comprises contacting the sample to the solid support. In some embodiments, the SiMREPS assay method further comprises identifying a candidate aptamer and introducing a number of single-nucleotide changes into the conserved target binding region of the candidate aptamer to produce the aptamer query probe. In some embodiments, identifying the candidate aptamer comprises using in vitro evolution (e.g., SELEX). In some embodiments, producing the aptamer query probe comprises truncating the candidate aptamer.

In some embodiments, recording the time dependent change in the signal intensity of the detectable label comprises recording a series of images. In some embodiments, the SiMREPS assay method further comprises producing an intensity fluctuation map by determining an average absolute image-to-image change in intensity at a number of image pixels. In some embodiments, the SiMREPS assay method further comprises generating intensity versus-time data and calculating a kinetic parameter from the intensity versus-time data. In some embodiments, the SiMREPS assay method further comprises identifying positive detection events using a threshold for the kinetic parameter.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A to 1D show detection of single protein molecules by aptamer-based kinetic fingerprinting. FIG. 1A shows an exemplary experimental scheme for detecting a target protein by aptamer-based SiMREPS. FIG. 1B shows the sequences and secondary structures of aptamers for detecting IL-8 (aptamers 8A-44, 8A-35, 8A-30) and VEGF₁₆₅ (aptamer t22). FIG. 1C shows single movie frames of representative microscope FOV and FIG. 1D shows intensity-versus time traces showing the distinct kinetic fingerprints of specific, repetitive binding to a target protein (top trace) and nonspecific binding to the assay surface (bottom trace). In the microscope frames, the bright puncta represent single fluorophore-labelled aptamers bound at or near the coverslip surface. The upper frame depicts a FOV from an experiment in the presence of recombinant IL-8 and the lower frame depicts a FOV from an experiment without IL-8.

FIG. 2A to 2G show aptamer based detection of VEGF₁₆₅. FIG. 2A shows that a G-to-A mutation (shown in red and bold-faced) introduced into the conserved region (boxed) of the t22 aptamer results in the more rapidly dissociating t22 G→A aptamer. FIG. 2B shows representative kinetic time traces of interaction of VEGF₁₆₅ with the t22 aptamer (upper trace) and the t22 G→A aptamer (lower trace). FIG. 2C shows the cumulative bound and unbound (inset) dwell time histograms of the Cy5-labelled t22 aptamer (blue squares) and t22 G→A aptamer (red circles). The overall binding (kₒₙ) and dissociation (k_{off}) rate constants of interaction between VEGF₁₆₅ and t22 or between VEGF₁₆₅ and t22 G→A are indicated. The reported errors are the standard error of the mean from two independent replicates. FIG. 2D to 2G are scatterplots of N_{b+d} and τ_{on, median} for all intensity versus-time trajectories observed within a single field of view in the absence (FIGS. 2D and 2F) and in the presence (FIGS. 2E and 2G) of 5 pM VEGF₁₆₅ (10 minutes acquisition time, 500 ms exposure, 1200 frames). FIG. 2D and FIG. 2E depict the kinetics of the original t22 aptamer; FIGS. 2F and 2G depict the kinetics of the modified t22 G→A aptamer. The dashed lines indicate the minimum and maximum thresholds for accepting a trace as evidence of a single VEGF₁₆₅ molecule. “+” symbols represent traces that do not pass filtering for intensity, signal to noise, and/or kinetics and are rejected as detection events of VEGF₁₆₅; red filled circles represent traces that pass filtering and are considered positive detection events.

FIG. 3A to 3C show single field of view showing fluorescent spots density on the surface in presence of 5 pM VEGF₁₆₅ (FIG. 3A) and in absence of VEGF₁₆₅ (FIG. 3B). The number of fluorescent spots is approximately 8-10 times higher in the sample well containing VEGF₁₆₅ than the control well without VEGF₁₆₅ under identical buffer (1×PBS containing 100 nM Cy5 labelled t22 aptamer) and imaging condition (FIG. 3C). Intensity threshold for fluorescent spot counting is I_{threshold}=600 and signal to noise ratio, S/N=2.

FIG. 4A to 4C show scatterplots of N_{b+d} and τ_{on, median} for all intensity versus-time trajectories observed within a single field of view in the absence of VEGF₁₆₅ (FIG. 4A) and in the presence (FIG. 4B and FIG. 4C) of 5 pM VEGF₁₆₅. The imaging solution for A and B contains 1×PBS and that for C contains 6×PBS. Points indicated by “+” represent traces that do not pass filtering for intensity, signal-to-noise, and/or kinetics and are not considered as interaction between VEGF₁₆₅ and aptamers. Points indicated by red filled circles represent traces that pass filtering and are considered positive detection events.

FIG. 5A and FIG. 5B show scatterplots of N_{b+d} and τ_{on, median} for all intensity-versus time trajectories observed within a single field of view in the absence of VEGF₁₆₅ (FIG. 5A) and in the presence (FIG. 5B) of 60 pM biotinylated VEGF₁₆₅. The imaging solution for A and B contains 6×PBS and the data were collected at 36° C. Points indicated by “+” represent traces that do not pass filtering for intensity, signal-to-noise, and/or kinetics and are not considered as interaction between VEGF₁₆₅ and aptamers. Points indicated by red filled circles represent traces that pass filtering and are considered positive detection events.

FIG. 6A to 6D show modified aptamers of t22. The mutated bases are shown in bold red color. The scatterplots below each aptamer show the N_{b+d} and τ_{on, median} for all intensity versus-time trajectories observed for the aptamer within a single field of view in the presence of 5 pM VEGF₁₆₅ in 6×PBS at room temperature. The modified aptamers shown in FIG. 6A to 6C associate with VEGF₁₆₅. However, mutation of G to A at the position shown in FIG. 6D eliminates and/or makes non-detectable the interaction of the aptamer with VEGF₁₆₅.

FIG. 7A to 7I show characterization of aptamer probes for IL-8. FIG. 7A shows a schematic depiction of two capture strategies for IL-8: direct capture of C-terminally biotinylated IL-8 to streptavidin; and antibody-mediated capture of non-biotinylated IL-8. FIG. 7B and FIG. 7C show dissociation rate constants (FIG. 7B) and association rate constants (FIG. 7C) of Cy5 labelled aptamers (8A-44, 8A-35, and 8A-30) interacting with directly captured biotinylated II. 8 as measured by TIRF microscopy. FIG. 7D and FIG. 7E show dissociation rate constants (FIG. 7D) and association rate constants (FIG. 7E) of Cy5-labelled aptamers interacting with antibody captured IL-8. Error bars represent the standard error of the mean from two independent replicates. FIG. 7F and FIG. 7G show normalized histograms of the number of intensity transitions per single-molecule trace arising from aptamer binding or dissociation (N_{b+d}) of Cy5-labelled aptamers interacting with directly captured biotinylated IL-8 (FIG. 7F) or antibody captured IL-8 (FIG. 7G) with a 5-min acquisition time at 21° C. FIG. 7H and FIG. 7I show scatterplots of N_{b+d} and τ_{on, median} for all intensity versus-time trajectories observed within a single field of view in the presence (FIG. 7H) and in the absence (FIG. 7I) of 1 pM IL-8 (1 minute acquisition time at 24° C.). Dashed lines indicate thresholds for accepting a trace as evidence of a single IL-8 molecule. Points indicated by “+” represent traces that do not pass filtering for intensity, signal to noise, and/or kinetics and are not considered as interactions between IL-8 and 8A-30 aptamer. Points indicated by red filled circles represent traces that pass filtering and are considered positive detection events.

FIG. 8A and FIG. 8B show representative kinetic time traces of the 8A aptamer interaction with IL-8. FIG. 8A shows the interaction of 8A aptamers with directly surface-captured biotinylated IL-8. FIG. 8B shows interactions of 8A aptamers with monoclonal antibody captured IL-8.

FIG. 9A to 9D show scatterplots of N_{b+d} and τ_{on, median} for all intensity versus-time trajectories observed within a single field of view in the absence of IL-8 (FIGS. 9A and 9C) and in the presence of 10 pM IL-8 in 25% horse serum (FIG. 9B and FIG. 9D) using a 2 minute acquisition time at 24° C. FIG. 9A and FIG. 9B show data from experiments using the 8A-44 probe; FIG. 9C and FIG. 9D show data from experiments using the 8A-30 probe. Dashed lines indicate thresholds for accepting a trace as evidence of a single IL-8 molecule. Points indicated by “+” represent traces that do not pass filtering for intensity, signal-to-noise, and/or kinetics and are not considered as interaction between IL-8 and 8A aptamers. Points indicated by red filled circles represent traces that pass filtering and are considered positive detection events.

FIG. 10A to FIG. 10C show the influence of temperature on the interaction kinetics of 8A-30 aptamer with IL-8. FIG. 10A shows that the peak of the N_{b+d} distribution shifts towards higher values by increasing the temperature from 21° C. to 24° C. A further increase in temperature does not further affect the peak. FIG. 10B shows that the aptamer bound time and hence the dissociation rates are similar at different temperatures. FIG. 10C show that the association rate increases with an increase in temperature from 21° C. to 24° C. However, increasing the temperature further to 27° C. only changes the shape of the cumulative distribution curve.

FIG. 11A to FIG. 11E show wash-free assays of spiked in and endogenous protein targets. FIG. 11A shows a wash-free SiMREPS protocol for quantifying IL-8 and VEGF₁₆₅ in serum. A serum sample containing spiked-in or endogenous IL-8 or VEGF₁₆₅ was combined with the imaging solution and then added to a capture antibody-coated coverslip. After a suitable incubation period (40 minutes), the sample was imaged by TIRF microscopy to quantify IL-8 and VEGF₁₆₅. FIG. 11B and FIG. 11C are standard curves showing quantification of spiked-in VEGF₁₆₅ (FIG. 11B) or IL-8 (FIG. 11C) using aptamer-based SiMREPS (blue squares) or sandwich ELISA (orange circles). Linear regression fits are shown as blue (SiMREPS) or orange (sandwich ELISA) solid lines. Error bars indicate the SD of three independent measurements. FIG. 11D shows scatterplots of N_{b+d} and τ_{on, median} for all intensity versus time trajectories observed within a single field of view for the detection of endogenous IL-8 from 2% human serum at 24° C. FIG. 11E shows that SiMREPS detects IL-8 in 2% and 10% human serum (blue square); by contrast, a commercial ELISA (orange circle) cannot detect IL-8 even in undiluted (100%) human serum.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detecting analytes and particularly, but not exclusively, to methods, compositions, systems, and kits for detecting analytes using aptamer technologies.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., an analyte). For example, when an analyte is said to be “present” in a test sample, it means the level or amount of this analyte is above a pre determined threshold; conversely, when an analyte is said to be “absent” in a test sample, it means the level or amount of this analyte is below a pre determined threshold. The pre determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Another relative change indicating an “increase” or “decrease” is a change in a measured value that is at least 2 or 3 times the standard deviation of background noise. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

As used herein, the terms “subject” and “patient” refer to any organisms including plants, microorganisms, and animals (e.g., mammals such as dogs, cats, livestock, and humans).

The term “sample” in the present specification and claims is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids (a “biofluid”), solids, tissues, and gases. Environmental samples include environmental material such as surface matter, soil, water, and industrial samples. These examples are not to be construed as limiting the sample types applicable to the present technology.

As used herein, a “biological sample” refers to a sample of biological tissue or fluid (a “biofluid”). For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non disposable items. These examples are not to be construed as limiting the sample types applicable to the present invention.

The term “label” as used herein refers to any atom, molecule, molecular complex (e.g., metal chelate), or colloidal particle (e.g., quantum dot, nanoparticle, microparticle, etc.) that can be used to provide a detectable (preferably quantifiable) effect, and that can be attached to a nucleic acid or protein. Labels include, but are not limited to, dyes (e.g., optically detectable labels, fluorescent dyes or moieties, etc.); radiolabels such as ³²P; binding moieties such as biotin; haptens such as digoxgenin; luminogenic, phosphorescent, optically-detectable, or fluorogenic moieties; mass tags; and fluorescent dyes alone or in combination with moieties that can suppress or shift emission spectra by fluorescence resonance energy transfer (FRET). Labels may provide signals detectable by fluorescence, luminescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein, the term “support” or “solid support” refers to a matrix on or in which an analyte, capture probe, and the like may be immobilized, e.g., to which they may be covalently or noncovalently attached or in or on which they may be partially or completely embedded so that they are largely or entirely prevented from diffusing freely or moving with respect to one another.

As used herein, “moiety” refers to one of two or more parts into which something may be divided, such as, for example, the various parts of an oligonucleotide, a molecule, a chemical group, a domain, a probe, a polypeptide, etc.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double stranded, and represent the sense or antisense strand.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

As used herein, the term “peptide nucleic acid” refers to a DNA mimic that incorporates a peptide-like polyamide backbone.

As used herein, the terms “complementary” or “complementarity” are used in reference to polynucleotides (e.g., a sequence of nucleotides such as an oligonucleotide capture probe, query probe or a target analyte that is a nucleic acid) related by the base-pairing rules. For example, for the sequence “5′-A-G-T-3” is complementary to the sequence “3′T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, the term “mismatch” means a nucleobase of a first nucleic acid that is not capable of pairing with a nucleobase at a corresponding position of a second nucleic acid.

As used herein, the term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include transmembrane domains, glycosylation sites, etc.

As used herein, the term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

As used herein, the term “wild type” refers to a gene or a gene product that has the characteristics of that gene or gene product when isolated from a naturally occurring source. A wild type gene is that which is most frequently observed in a population and is thus arbitrarily designated the “normal” or “wild type” form of the gene. In contrast, the term “modified,” “mutant,” or “polymorphic” refers to a gene or gene product that displays modifications in sequence and or functional properties (i.e., altered characteristics) when compared to the wild type gene or gene product. It is noted that naturally occurring mutants can be isolated; these are identified by the fact that they have altered characteristics when compared to the wild type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; in some embodiments, one sequence is a reference sequence.

As used herein, the term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

As used herein, the term “Tₘ” is used in reference to the “melting temperature.” The melting temperature is the temperature at which a population of double stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see, e.g., Anderson and Young, Quantitative Filter Hybridization, in Nucleic Acid Hybridization (1985). Other references (e.g., Allawi and SantaLucia, Biochemistry 36: 10581-94 (1997) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

As used herein, the terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one and three-letter amino acid codes are used herein as follows-Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

As used herein, the terms “variant” and “mutant” when used in reference to a polypeptide refer to an amino acid sequence that differs by one or more amino acids from another, usually related polypeptide.

As used herein, the term “melting” when used in reference to a nucleic acid refers to the dissociation of a double-stranded nucleic acid or region of a nucleic acid into a single-stranded nucleic acid or region of a nucleic acid.

As used herein, a “query probe” or “reader probe” is any entity (e.g., molecule, biomolecule, etc.) that recognizes an analyte (e.g., binds to an analyte, e.g., binds specifically to an analyte). In exemplary embodiments, the query probe is a protein that recognizes an analyte. In some other exemplary embodiments, the query probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, the query probe is labeled, e.g., with a detectable label such as, e.g., a fluorescent moiety as described herein. In some embodiments, the query probe comprises more than one type of molecule (e.g., more than one of a protein, a nucleic acid, a chemical linker or a chemical moiety).

As used herein, an “event” refers to an instance of a query probe binding to an analyte or an instance of query probe dissociation from an analyte, e.g., as measured by monitoring a detectable property indicating the binding of a query probe to an analyte and/or the dissociation of a query probe from an analyte.

As used herein, the term “N_{b+d}” refers to the number of binding (b) and dissociation (d) events of one or more detectably labeled query probes (e.g., aptamer query probes) observed at a single location (e.g., a discrete region of a solid support, a single diffraction-limited region of an image, or a single location of a specimen as determined by super-resolution imaging) within a defined time window of observation (e.g., 10 seconds, 20 seconds, 30 seconds, 1 minute, . . . , etc.) N_{b+d} is determined by counting the number of sudden (e.g., within a span of time smaller than the time resolution of the measurement, e.g., less than approximately 500 milliseconds or less than approximately 100 milliseconds) increases (corresponding to binding events) and decreases (corresponding to dissociation events) in query probe signal (e.g., fluorescence intensity) at a single location within the observation window. The counting of binding and dissociation events may be performed using one or more of several different algorithms, including but not limited to: edge detection algorithms, hidden Markov models, super resolution analysis of intensity difference maps, k-means clustering, least-squares fitting, machine learning, or deep learning.

As used herein, the term “τ_{on, median}” (also known as “τ_{bound, median}”) refers to the median value of the apparent residence times of all detectably labeled query probes observed at a single location (e.g., a discrete region of a solid support, a single diffraction limited region of an image, or a single location of a specimen as determined by super-resolution imaging) within a defined time window of observation (e.g., 10 seconds, 20 seconds, 30 seconds, 1 minute, . . . , etc.)

As used herein, a “capture probe” is any entity (e.g., molecule, biomolecule, etc.) that recognizes an analyte (e.g., binds to an analyte, e.g., binds specifically to an analyte) and links the analyte to a solid support. In exemplary embodiments, the capture probe is a protein that recognizes an analyte. In some other exemplary embodiments, a capture probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, a capture probe is labeled, e.g., with a detectable label such as, e.g., a fluorescent moiety as described herein. In some embodiments, the capture probe comprises more than one type of molecule (e.g., more than one of a protein, a nucleic acid, a chemical linker or a chemical moiety). In some embodiments, the analyte is modified (e.g., with a ligand moiety) and the capture probe specifically binds the ligand moiety. A non-limiting example of a ligand moiety is a biotinyl group linked to the analyte and a non-limiting example of the associated specific capture probe that specifically binds to the biotinylated analyte is a streptavidin moiety.

As used herein, the term “sensitivity” refers to the probability that an assay gives a positive result for the analyte when the sample comprises the analyte. Sensitivity is calculated as the number of true positive results divided by the sum of the true positives and false negatives. Sensitivity is a measure of how well an assay detects an analyte.

As used herein, the term “specificity” refers to the probability that an assay gives a negative result when the sample does not comprise the analyte. Specificity is calculated as the number of true negative results divided by the sum of the true negatives and false positives. Specificity is a measure of how well a method of the present invention excludes samples that do not comprise an analyte from those that do comprise the analyte.

As used herein, the “equilibrium constant” (K_{eq}), the “equilibrium association constant” (Kₐ), and “association binding constant” (or “binding constant” (K_{B})) are used interchangeably for the following binding reaction of A and B at equilibrium:

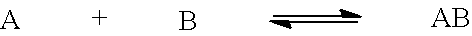

where A and B are two entities that associate with each other (e.g., capture probe and analyte, query probe and analyte) and K_{eq}=[AB]/([A]×[B]). The dissociation constant K_{D}=1/K_{B}. The K_{D} is a useful way to describe the affinity of a one binding partner A for a partner B with which it associates, e.g., the number K_{D} represents the concentration of A or B that is required to yield a significant amount of AB. K_{eq}=k_{off}/kₒₙ; K_{D}=k_{off}/kₒₙ.

As used herein, a “significant amount” of the product of two entities that associate with each other, e.g., formation of AB from A and B according to the equation above, refers to a concentration of AB that is equal to or greater than the free concentration of A or B, whichever is smaller.

As used herein, “nanomolar affinity range” refers to the association of two components that has an equilibrium dissociation constant K_{D} (e.g., ratio of k_{off}/kₒₙ) in the nanomolar range, e.g., a dissociation constant (K_{D}) of 1×10⁻¹⁰ to 1×10⁻⁵ M (e.g., in some embodiments 1×10⁻⁹ to 1×10⁻⁶ M). The dissociation constant has molar units (M). The smaller the dissociation constant, the higher the affinity between two components (e.g., capture probe and analyte; query probe and analyte).

As used herein, a “weak affinity” or “weak binding” or “weak association” refers to an association having a K_{D} of approximately 100 nanomolar (e.g., approximately 10, 20, 30, 40, 50, 60, 70, 80, 90, 100, 110, 120, 130, 140, 150, 160, 170, 180, 190, 200, 300, 400, or 500 nanomolar) and/or, in some embodiments, in the range of 1 nanomolar to 10 micromolar.

The terms “specific binding” or “specifically binding” when used in reference to the interaction of two components A and B that associate with one another refers to an association of A and B having a K_{D} that is smaller than the K_{D} for the interaction of A or B with other similar components in the solution, e.g., at least one other molecular species in the solution that is not A or B.

The term “detection assay” refers to an assay for detecting the presence or absence of an analyte or the activity or effect of an analyte or for detecting the presence or absence of a variant of an analyte.

In some embodiments the technology comprises an antibody component or moiety, e.g., an antibody or fragments or derivatives thereof. As used herein, an “antibody”, also known as an “immunoglobulin” (e.g., IgG, IgM, IgA, IgD, IgE), comprises two heavy chains linked to each other by disulfide bonds and two light chains, each of which is linked to a heavy chain by a disulfide bond. The specificity of an antibody resides in the structural complementarity between the antigen combining site of the antibody (or paratope) and the antigen determinant (or epitope). Antigen combining sites are made up of residues that are primarily from the hypervariable or complementarity determining regions (CDRs). Occasionally, residues from nonhypervariable or framework regions influence the overall domain structure and hence the combining site. Some embodiments comprise a fragment of an antibody, e.g., any protein or polypeptide containing molecule that comprises at least a portion of an immunoglobulin molecule such as to permit specific interaction between said molecule and an antigen. The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof. Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques, or can be prepared as a contiguous protein using genetic engineering techniques.

Fragments of antibodies include, but are not limited to, Fab (e.g., by papaine digestion), F(ab′)₂ (e.g., by pepsin digestion), Fab′ (e.g., by pepsin digestion and partial reduction) and Fv or scFv (e.g., by molecular biology techniques) fragments.

A Fab fragment can be obtained by treating an antibody with the protease papaine. Also, the Fab may be produced by inserting DNA encoding a Fab of the antibody into a vector for prokaryotic expression system or for eukaryotic expression system, and introducing the vector into a prokaryote or eukaryote to express the Fab. A F(ab′)₂ may be obtained by treating an antibody with the protease pepsin. Also, the F(ab′)₂ can be produced by binding a Fab′ via a thioether bond or a disulfide bond. A Fab may be obtained by treating F(ab′)₂ with a reducing agent, e.g., dithiothreitol. Also, a Fab′ can be produced by inserting DNA encoding a Fab′ fragment of the antibody into an expression vector for a prokaryote or an expression vector for a eukaryote and introducing the vector into a prokaryote or eukaryote for its expression. A Fv fragment may be produced by restricted cleavage by pepsin, e.g., at 4° C. and pH 4.0. (a method called “cold pepsin digestion”). The Fv fragment consists of the heavy chain variable domain (V_{H}) and the light chain variable domain (V_{L}) held together by strong noncovalent interaction. A scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv.

In general, antibodies can usually be raised to any antigen, using the many conventional techniques now well known in the art.

As used herein, the term “conjugated” refers to when one molecule or agent is physically or chemically coupled or adhered to another molecule or agent. Examples of conjugation include covalent linkage and electrostatic complexation. The terms “complexed,” “complexed with,” and “conjugated” are used interchangeably herein.

As used herein, a “stable interaction” or referring to a “stably bound” interaction refers to an association that is relatively persistent under the thermodynamic equilibrium conditions of the interaction. In some embodiments, a “stable interaction” is an interaction between two components having a K_{D} that is smaller than approximately 10⁻⁹ M or, in some embodiments a K_{D} that is smaller than 10⁻⁸ M. In some embodiments, a “stable interaction” has a dissociation rate constant k_{off} that is smaller than 1 per hour or, in some embodiments, a dissociation rate constant k_{off} that is smaller than 1 per minute. In some embodiments, a “stable interaction” is defined as not being a “transient interaction”. In some embodiments, a “stable interaction” includes interactions mediated by covalent bonds and other interactions that are not typically described by a K_{D} value but that involve an average association lifetime between two entities that is longer than approximately 1 minute (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 seconds) per each interaction.

In some embodiments, the distinction between a “stable interaction” and a “transient interaction” is determined by a cutoff value of K_{D} and/or k_{off} and/or another kinetic or thermodynamic value describing the associations, wherein the cutoff is used to discriminate between stable and transient interactions that might otherwise be characterized differently if described in absolute terms of a K_{D} and/or k_{off} or another kinetic or thermodynamic value describing the associations. For example, a “stable interaction” characterized by a K_{D} value might also be characterized as a “transient interaction” in the context of another interaction that is even more stable. One of skill in the art would understand other relative comparisons of stable and transient interactions, e.g., that a “transient interaction” characterized by a K_{D} value might also be characterized as a “stable interaction” in the context of another interaction that is even more transient (less stable).

As used herein, “moiety” refers to one of two or more parts into which something may be divided, such as, for example, the various parts of an oligonucleotide, a molecule, a chemical group, a domain, a probe, an “R” group, a polypeptide, etc.

As used herein, in some embodiments a “signal” is a time varying quantity associated with one or more properties of a sample that is assayed, e.g., the binding of a query probe to an analyte and/or dissociation of a query probe from an analyte. A signal can be continuous in the time domain or discrete in the time domain. As a mathematical abstraction, the domain of a continuous time signal is the set of real numbers (or an interval thereof) and the domain of a discrete-time signal is the set of integers (or an interval thereof). Discrete signals often arise via “digital sampling” of continuous signals. For example, an audio signal consists of a continually fluctuating voltage on a line that can be digitized by reading the voltage level on the line at a regular interval, e.g., every 50 microseconds. The resulting stream of numbers is stored as a discrete-time digital signal. In some embodiments, the signal is recorded as a function of location is space (e.g., x, y coordinates; e.g., x, y, z coordinates). In some embodiments, the signal is recorded as a function of time. In some embodiments, the signal is recorded as a function of time and location.

The term “substantially” as used herein, is a broad term and is used in its ordinary sense, including, but not limited to, being largely but not necessarily wholly that which is specified.

The term “algorithm,” as used herein, is a broad term and is used in its ordinary sense, including, but not limited to, the computational processes (for example, programs) involved in transforming information from one state to another, for example using computer processing.

### DESCRIPTION

Although the disclosure herein refers to certain illustrated embodiments, it is to be understood that these embodiments are presented by way of example and not by way of limitation.

### Poisson Processes

Embodiments of the technology are related to single molecule recognition by recording the characteristic kinetics of a query probe binding to a target analyte. In particular embodiments, this process is a Poisson process. A Poisson process is a continuous-time stochastic process that counts the number of events and the time that events (e.g., transient binding of a detectably labeled (e.g., fluorescent) query probe to an immobilized target analyte) occur in a given time interval. The time interval between each pair of consecutive events has an exponential distribution and each interval is assumed to be independent of other intervals. The Poisson distribution is a discrete probability distribution that expresses the probability of a given number of the events occurring in the given time interval if these events occur with a known average rate and independently of the time since the last event. The Poisson distribution can also be used for the number of events in other specified intervals such as distance, area, or volume.

A Poisson distribution is a special case of the general binomial distribution where the number of trials n is large, the probability of success p is small, and the product np=λ is moderate. In a Poisson process, the probability that a number of events N is j at any arbitrary time t follows the Poisson probability distribution Pⱼ(t).

$${P}_{j} ( t ) = \frac{{{e}^{- λ t} ( λ t )}^{j}}{j !} , j = 0 , 1 , 2 , … . \qquad ( 1 )$$

That is, the number N of events that occur up to time t has a Poisson distribution with parameter λt. Statistical and mathematical methods relevant to Poisson processes and Poisson distributions are known in the art. See, e.g., “Stochastic Processes (i): Poisson Processes and Markov Chains” in Statistics for Biology and Health—Statistical Methods in Bioinformatics (Ewans and Grant, eds.), Springer (New York, 2001), page 129 et seq., incorporated herein by reference in its entirety. Software packages such as Matlab and R may be used to perform mathematical and statistical methods associated with Poisson processes, probabilities, and distributions.

### Kinetics of Detection

Particular embodiments of the technology are related to detecting an analyte by analyzing the kinetics of the interaction of a query probe with the analyte to be detected. For the interaction of a query probe Q (e.g., at an equilibrium concentration [Q]) with a target analyte T (e.g., at an equilibrium concentration [T]), the kinetic rate constant kₒₙ describes the time-dependent formation of the complex QT comprising the probe Q hybridized to the analyte T. In particular embodiments, while the formation of the QT complex is associated with a second order rate constant that is dependent on the concentration of query probe and has units of M⁻¹min⁻¹ (or the like), the formation of the QT complex is sufficiently described by a kₒₙ that is a pseudo-first order rate constant associated with the formation of the QT complex. Thus, in some embodiments, kₒₙ is an apparent (“pseudo”) first-order rate constant.

Likewise, the kinetic rate constant k_{off} describes the time-dependent dissociation of the complex QT into the probe Q and the analyte T. Kinetic rates are typically provided herein in units of min⁻¹ or s⁻¹. The “dwell time” of the query probe Q in the bound state (τₒₙ) is the time interval (e.g., length of time) that the probe Q is hybridized to the analyte T during each instance of query probe Q binding to the analyte T to form the QT complex. The “dwell time” of the query probe Q in the unbound state (τ_{off}) is the time interval (e.g., length of time) that the probe Q is not hybridized to the analyte T between each instance of query probe Q binding to the analyte to form the QT complex (e.g., the time the query probe Q is dissociated from the target analyte T between successive binding events of the query probe Q to the target analyte T). Dwell times may be provided as averages or weighted averages integrating over numerous binding and non-binding events.

Further, in some embodiments, the repeated, stochastic binding of probes (e.g., detectably labeled query probes (e.g., fluorescent probes) to target analytes is modeled as a Poisson process occurring with constant probability per unit time and in which the standard deviation in the number of binding and dissociation events per unit time (N_{b+d}) increases as (N_{b+d})^{1/2}. Thus, the statistical noise becomes a smaller fraction of N_{b+d} as the observation time is increased. Accordingly, the observation is lengthened as needed in some embodiments to achieve discrimination between target and off-target binding. And, as the acquisition time is increased, the signal and background peaks in the N_{b+d} histogram become increasingly separated and the width of the signal distribution increases as the square root of N_{b+d}, consistent with kinetic Monte Carlo simulations.

Further, in some embodiments assay conditions are controlled to tune the kinetic behavior to improve discrimination of query probe binding events to the target analyte from background binding. For example, in some embodiments the technology comprises control of assay conditions such as, e.g., using a query probe that is designed to interact weakly with the target analyte (e.g., in the nanomolar affinity range); controlling the temperature such that the query probe interacts weakly with the target analyte; controlling the solution conditions, e.g., ionic strength, ionic composition, addition of chaotropic agents, addition of competing probes, and/or addition of molecular crowding agents.

### Analytes

The technology is not limited in the analyte that is detected, quantified, identified, or otherwise characterized (e.g., presence, absence, amount, concentration, state). The term “analyte” as used herein is a broad term and is used in its ordinary sense, including, without limitation, to refer to a substance or chemical constituent in a sample such as a biological fluid (for example, blood, interstitial fluid, cerebral spinal fluid, lymph fluid or urine) that can be analyzed. In some embodiments, samples comprise multiple substances or chemical constituents that are of the same and/or different types. Accordingly, in some embodiments, the terms “target analyte” and “non target analyte” are used to differentiate an analyte that is the object of detection, quantification, identification, or characterization (e.g., presence, absence, amount, concentration, state) by an assay from other substance or chemical constituents that may be the same type as the analyte but which are not the object of detection, quantification, identification, or characterization (e.g., presence, absence, amount, concentration, state) by the assay. Thus, when used herein, the term “target analyte” refers to an analyte that is the object of detection, quantification, identification, or characterization (e.g., presence, absence, amount, concentration, state) by an assay. The term “non target analyte” refers to a substance or chemical constituent of a sample that is not the object of detection, quantification, identification, or characterization (e.g., presence, absence, amount, concentration, state) by an assay. The “non target analyte” may or may not be the same type of substance or chemical constituent as the analyte.

Analytes can include naturally occurring substances, artificial substances, metabolites, and/or reaction products. In some embodiments, the analyte comprises a salt, sugars, protein, fat, vitamin, or hormone. In some embodiments, the analyte is naturally present in a biological sample (e.g., is “endogenous”); for example, in some embodiments, the analyte is a metabolic product, a hormone, an antigen, an antibody, and the like. Alternatively, in some embodiments, the analyte is introduced into a biological organism (e.g., is “exogenous”), for example, a drug, drug metabolite, a drug precursor (e.g., prodrug), a contrast agent for imaging, a radioisotope, a chemical agent, etc. The metabolic products of drugs and pharmaceutical compositions are also contemplated analytes.

In some embodiments, the analyte is a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a combination of one or more of a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc.

In some embodiments, the analyte is part of a multimolecular complex, e.g., a multiprotein complex, a nucleic acid/protein complex, a molecular machine, an organelle (e.g., a cell-free mitochondrion, e.g., in plasma; a plastid; golgi, endoplasmic reticulum, vacuole, peroxisome, lysosome, and/or nucleus), cell, virus particle, tissue, organism, or any macromolecular complex or structure or other entity that can be captured and/or detected and that is amenable to analysis by the technology described herein (e.g., a ribosome, spliceosome, vault, proteasome, DNA polymerase III holoenzyme, RNA polymerase II holoenzyme, symmetric viral capsids, GroEL/GroES; membrane protein complexes: photosystem I, ATP synthase, nucleosome, centriole and microtubule-organizing center (MTOC), cytoskeleton, flagellum, nucleolus, stress granule, germ cell granule, or neuronal transport granule). For example, in some embodiments, a multimolecular complex is isolated and the technology finds use in characterizing, identifying, quantifying, and/or detecting one or more molecules (analytes) associated with (e.g., that is a component of) the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

In some embodiments, the analyte is chemically modified to provide a site for query probe binding. For instance, in some embodiments, beta-elimination of phosphoserine and phosphothreonine under strongly basic conditions is used to introduce an alkene, followed by Michael addition of a nucleophile such as a dithiol to the alkene. The remaining free thiol is then used for conjugation to a maleimide-containing oligonucleotide with a sequence complementary to an oligonucleotide query probe. The post translational modifications phosphoserine and phosphothreonine may then be probed using the query probe and analyzed as described herein.

In some embodiments, the analyte is modified to comprise a moiety that is specifically bound by a capture probe.

As used herein “detect an analyte” or “detect a substance” will be understood to encompass direct detection of the analyte itself or indirect detection of the analyte by detecting its by-product(s).

### Capture

Embodiments of the technology comprise capture of an analyte. In some embodiments, the analyte is captured and immobilized. In some embodiments, the analyte is stably attached to a solid support. In some embodiments, the solid support is immobile relative to a bulk liquid phase contacting the solid support. In some embodiments, the solid support is diffusible within a bulk liquid phase contacting the solid support.

In some embodiments, stable attachment of the target analyte to a surface or other solid substrate is provided by a high affinity or irreversible interaction (e.g., as used herein, an “irreversible interaction” refers to an interaction having a dissociation half-life longer than the observation time, e.g., in some embodiments, a time that is 1 to 5 minutes (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer). The technology is not limited in the components and/or methods used for capture of the analyte. For example, the stable attachment is provided by a variety of methods, including but not limited to one or more of the following.

In some embodiments, an analyte is immobilized by a surface-bound capture probe with a dissociation constant (K_{D}) for the analyte smaller than approximately 1 nanomolar (nM) (e.g., less than 1.5, 1.4, 1.3, 1.2, 1.1, 1.0, 0.9, 0.8, 0.7, 0.6, 0.5 nanomolar) and a dissociation rate constant for the analyte that is smaller than approximately 1 min⁻¹ (e.g., less than approximately 1.5, 1.4, 1.3, 1.2, 1.1, 1.0, 0.9, 0.8, 0.7, 0.6, 0.5 min⁻¹). Exemplary surface-bound capture probes include, e.g., an antibody, antibody fragment, nanobody, or other protein; a high affinity DNA binding protein or ribonucleoprotein complex such as Cas9, dCas9, Cpf1, transcription factors, or transcription activator-like effector nucleases (TALENs); a nucleic acid such as a double-stranded oligonucleotide, a single stranded oligonucleotide, an aptamer; a small organic molecule; or a metal ion complex.

In some embodiments, an analyte is immobilized by direct noncovalent attachment to a surface (e.g., by interactions between the analyte and the surface, e.g., a glass surface or a nylon, nitrocellulose, or polyvinylidene difluoride membrane).

In some embodiments, an analyte is immobilized by chemical linking (e.g., by a covalent bond) of the analyte to the solid support. In some embodiments, the analyte is chemically linked to the solid support by, e.g., a carbodiimide, a N-hydroxysuccinimide esters (NHS) ester, a maleimide, a haloacetyl group, a hydrazide, or an alkoxyamine. In some embodiments, an analyte is immobilized by radiation (e.g., ultraviolet light)-induced cross-linking of the target analyte to the surface and/or to a capture probe attached to the surface. In some embodiments, the capture probe is a rabbit monoclonal antibody. In some embodiments in which the analyte comprises a carbohydrate or polysaccharide, the capture probe comprises a carbohydrate binding protein such as a lectin or a carbohydrate-binding antibody.

Alternatively, instead of immobilizing the target analyte to a solid support that is relatively stationary with respect to a bulk phase that contacts the solid support as described above, some embodiments provide that the target analyte is associated with a freely diffusing particle that diffuses within the bulk fluid phase contacting the freely diffusing particle. Accordingly, in some embodiments, the target analyte is covalently or noncovalently bound to a freely diffusing substrate. In some embodiments, the freely diffusing substrate is, e.g., a colloidal particle (e.g., a particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)). In some embodiments, the freely diffusing substrate comprises and/or is made of, e.g., polystyrene, silica, dextran, gold, or DNA origami. In some embodiments, the target analyte is associated with a freely diffusing particle that diffuses slowly relative to the diffusion of the query probe, e.g., the target analyte has a diffusion coefficient that is less than approximately 10% (e.g., less than 15, 14, 13, 12, 11, 10.5, 10.4, 10.3, 10.2, 10.1, 10.0, 9.9, 9.8, 9.7, 9.6, 9.5, or 9.0% or less) of the diffusion coefficient of the query probe.

Furthermore, in some embodiments the target analyte is associated with a freely diffusing particle and the location of the target analyte is observable and/or recordable independently of observing and/or recording query probe binding. For example, in some embodiments a detectable label (e.g., a fluorophore, fluorescent protein, quantum dot) is covalently or noncovalently attached to the target analyte, e.g., for detection and localization of the target analyte. Accordingly, in some embodiments the position of the target analyte and the position of query probe binding events are simultaneously and independently measured.

### Query

Embodiments of the technology comprise a query probe (e.g., a detectably labeled query probe) that binds transiently and repeatedly to the analyte, e.g., a query probe that binds to and dissociates from the target analyte several (e.g., greater than 1, 2, 3, 4, 5, 6, 7, 8, 9, 10, or more) times per observation window. In some embodiments, the query probe has a dissociation constant (K_{D}) for the analyte of larger than approximately 1 nanomolar (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 1.0, 4.1, 4.2, 4.3, 4.4, 4.5, 4.6, 4.7, 4.8, 4.9, or 5.0 or more nanomolar) under the assay conditions. In some embodiments, the query probe has a binding and/or a dissociation constant for the analyte that is larger than approximately 1 min¹ (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more min-1).

The technology is not limited in the query probe. In some embodiments, the query probe is an antibody or antibody fragment. In some embodiments, the query probe is a low-affinity antibody or antibody fragment. In some embodiments, the query probe is a nanobody, a DNA-binding protein or protein domain, a methylation binding domain (MBD), a kinase, a phosphatase, an acetylase, a deacetylase, an enzyme, or a polypeptide. In some embodiments, the query probe is an oligonucleotide that interacts with the target analyte. For example, in some embodiments the query probe is an oligonucleotide that hybridizes to the target analyte to form a duplex that has a melting temperature that is within approximately 10 degrees Celsius of the temperature at which the observations are made (e.g., approximately 7-12 nucleotides for observation that is performed at room temperature). In some embodiments, the query probe is an aptamer (e.g., as described herein). In some embodiments, the query probe is a fluorogenic probe. In some embodiments, the query probe is a mononucleotide.

In some embodiments, the query probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments, the query probe is a pharmaceutical agent, e.g., a drug or other bioactive molecule. In some embodiments, the query probe is a metal ion complex. In some embodiments, the query probe is a methyl-binding domain (e.g., MBD1). In some embodiments, the query probe is labeled with a detectable label as described herein. In some embodiments, the query probe is covalently linked to the detectable label. In some embodiments, the query probe is indirectly and/or non-covalently linked and/or associated with the detectable label. In some embodiments, the detectable label is fluorescent.

In some embodiments, the query probe is a fluorogenic probe comprising a detectable label (e.g., a fluorescent moiety) and a quencher of the detectable label.

In some embodiments, the query probe is a mouse monoclonal antibody.

In some embodiments in which the analyte comprises a carbohydrate or polysaccharide, the query probe comprises a carbohydrate binding protein such as a lectin or a carbohydrate-binding antibody.

In some embodiments, the query probe is a dye that binds to (e.g., associates with) an analyte. For example, in some embodiments, the query probe is a fluorogenic dye (e.g., a fluorescent dye that produces a fluorescent signal when associated with an analyte that is brighter than the fluorescent signal produced by the dye when dissociated from the analyte) that associates with an analyte and dissociates from the analyte with acceptable kinetics for use in SiMREPS technologies.

### Detection

The technology provides for the detection of target analytes, e.g., in the presence of similar analytes and, in some embodiments, background noise. In some embodiments, signal originating from the transient binding of the query probe to the target analyte is distinguishable from the signal produced by unbound query probe (e.g., by observing, monitoring, and/or recording a localized change in signal intensity during the binding event). In some embodiments, observing the transient binding of the query probe (e.g., a fluorescently labeled query probe) to the target analyte is provided by a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the technology provided herein uses query probes having a fluorescence emission that is quenched when not bound to the target analyte and/or a fluorescence emission that is dequenched when bound to the target analyte, e.g., a fluorogenic probe.

The technology comprises locating and/or observing the transient binding of a query probe to an analyte within a discrete region of an area and/or a discrete region of a volume that is observed, e.g., at particular spatial coordinates in a plane or a volume. In some embodiments, the error in determining the spatial coordinates of a binding or dissociation event (e.g., due to limited signal, detector noise, or spatial binning in the detector) is small (e.g., minimized, eliminated) relative to the average spacing between immobilized (e.g., surface-bound) target analytes. In some embodiments comprising use of wide-field fluorescence microscopy, measurement errors are minimized and/or eliminated by use of effective detector pixel dimensions in the specimen plane that are not larger than the average distance between immobilized (e.g., surface-bound) target analytes and that many fluorescent photons (in some embodiments, more than 100, e.g., more than 80, 85, 90, 95, 100, 105, 110, 115, 120, 125, or 130 or more) are collected per time point of detection.

In some embodiments, the detectable (e.g., fluorescent) query probe produces a fluorescence emission signal when it is close to the surface of the solid support (e.g., within about 100 nm of the surface of the solid support). When unbound, query probes quickly diffuse and thus are not individually detected; accordingly, when in the unbound state, the query probes produce a low level of diffuse background fluorescence. Consequently, in some embodiments detection of bound query probes comprises use of total internal reflection fluorescence microscopy (TIRF), HiLo microscopy (see, e.g., US20090084980, EP2300983 B1, WO2014018584 A1, WO2014018584 A1, incorporated herein by reference), confocal scanning microscopy, or other technologies comprising illumination schemes that illuminate (e.g., excite) only those query probe molecules near or on the surface of the solid support. Thus, in some embodiments, only query probes that are bound to an immobilized target near or on the surface produce a point-like emission signal (e.g., a “spot”) that can be confirmed as originating from a single molecule.

In some embodiments, the query probe comprises a fluorescent label having an emission wavelength. Detection of fluorescence emission at the emission wavelength of the fluorescent label indicates that the query probe is bound to an immobilized target analyte. Binding of the query probe to the target analyte is a “binding event”. In some embodiments of the technology, a binding event has a fluorescence emission having a measured intensity greater than a defined threshold. For example, in some embodiments a binding event has a fluorescence intensity that is above the background fluorescence intensity (e.g., the fluorescence intensity observed in the absence of a target analyte). In some embodiments, a binding event has a fluorescence intensity that is at least 1, 2, 3, 4 or more standard deviations above the background fluorescence intensity (e.g., the fluorescence intensity observed in the absence of a target analyte). In some embodiments, a binding event has a fluorescence intensity that is at least 2 standard deviations above the background fluorescence intensity (e.g., the fluorescence intensity observed in the absence of a target analyte). In some embodiments, a binding event has a fluorescence intensity that is at least 1.5, 2, 3, 4, or 5 times the background fluorescence intensity (e.g., the mean fluorescence intensity observed in the absence of a target analyte).

Accordingly, in some embodiments detecting fluorescence at the emission wavelength of the query probe that has an intensity above the defined threshold (e.g., at least 2 standard deviations greater than background intensity) indicates that a binding event has occurred (e.g., at a discrete location on the solid support where a target analyte is immobilized). Also, in some embodiments detecting fluorescence at the emission wavelength of the query probe that has an intensity above the defined threshold (e.g., at least 2 standard deviations greater than background intensity) indicates that a binding event has started. Accordingly, in some embodiments detecting an absence of fluorescence at the emission wavelength of the query probe that has an intensity above the defined threshold (e.g., at least 2 standard deviations greater than background intensity) indicates that a binding event has ended (e.g., the query probe has dissociated from the target analyte). The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the target analyte (e.g., an on/off event), e.g., a query probe dissociating from a bound state or a query probe associating with a target analyte from the unbound state.

Methods according to the technology comprise counting the number of query probe binding events that occur at each discrete location (e.g., at a position identified by x, y coordinates) on the solid support during a defined time interval that is the “acquisition time” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 0 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes).

Further, the length of time the query probe remains bound to the target analyte during a binding event is the “dwell time” of the binding event. The number of binding events detected during the acquisition time and/or the lengths of the dwell times recorded for the binding events is/are characteristic of a query probe binding to a target analyte and thus provide an indication that the target analyte is immobilized at said discrete location and thus that the target analyte is present in the sample.

Binding of the query probe to the immobilized target analyte and/or and dissociation of the query probe from the immobilized target analyte is/are monitored (e.g., using a light source to excite the fluorescent probe and detecting fluorescence emission from a bound query probe, e.g., using a fluorescence microscope) and/or recorded during a defined time interval (e.g., during the acquisition time). The number of times the query probe binds to the nucleic acid during the acquisition time and/or the length of time the query probe remains bound to the nucleic acid during each binding event and the length of time the query probe remains unbound to the nucleic acid between each binding event (e.g., the “dwell times” in the bound and unbound states, respectively) are determined, e.g., by the use of a computer and software (e.g., to analyze the data using a hidden Markov model and Poisson statistics).

In some embodiments, positive and/or negative control samples are measured (e.g., a control sample known to comprise or not to comprise a target). Fluorescence detected in a negative control sample is “background fluorescence” or “background (fluorescence) intensity” or “baseline”.

In some embodiments, data comprising measurements of fluorescence intensity at the emission wavelength of the query probe are recorded as a function of time. In some embodiments, the number of binding events and the dwell times of binding events (e.g. for each immobilized analyte) are determined from the data (e.g., by determining the number of times and the lengths of time the fluorescence intensity is above a threshold background fluorescence intensity). In some embodiments, transitions (e.g., binding and dissociation of a query probe) are counted for each discrete location on the solid support where a target analyte is immobilized. In some embodiments, a threshold number of transitions is used to discriminate the presence of a target analyte at a discrete location on the solid support from background signal, non target analyte, and/or spurious binding of the query probe.

In some embodiments, a distribution of the number of transitions for each immobilized target is determined—e.g., the number of transitions is counted for each immobilized analyte observed. In some embodiments a histogram is produced. In some embodiments, characteristic parameters of the distribution are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer.

Pattern recognition (e.g., using training sets, supervised learning, unsupervised learning, and analysis of unknown samples) associates identified patterns with analytes such that particular patterns (e.g., fluorescence intensity detected at one or more emission wavelengths as a function of time) provide a “kinetic fingerprint” of particular analytes that find use in detection, quantification, and identification of analytes.

In some embodiments, the distribution produced from a target analyte is significantly different than a distribution produced from a non target analyte or the distribution produced in the absence of a target analyte. In some embodiments, a mean number of transitions is determined for the plurality of immobilized target analytes. In some embodiments, the mean number of transitions observed for a sample comprising a target analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time at each discrete location on the solid support. In some particular embodiments, a relatively constant probability of a transition event occurring as a function of time at a discrete location on the solid support indicates the presence of a target analyte at said discrete location on the solid support. In some embodiments, a correlation coefficient relating event number and elapsed time is calculated from the probability of a transition event occurring as a function of time at a discrete location on the solid support. In some embodiments, a correlation coefficient relating event number and elapsed time greater than 0.95 when calculated from the probability of a transition event occurring as a function of time at a discrete location on the solid support indicates the presence of a target analyte at said discrete location on the solid support.

In some embodiments, dwell times of bound query probe (τₒₙ) and unbound query probe (τ_{off}) are used to identify the presence of an analyte in a sample and/or to distinguish a sample comprising a target analyte from a sample comprising a non target analyte and/or not comprising the analyte. For example, the τₒₙ for a target analyte is greater than the τₒₙ for a non-target analyte; and, the τ_{off} for a target analyte is smaller than the τ_{off} for a non-target analyte. In some embodiments, measuring τₒₙ and τ_{off} for a negative control and for a sample indicates the presence or absence of the analyte in the sample. In some embodiments, a plurality of τₒₙ and τ_{off} values is determined for each of a plurality of spots imaged on a solid support, e.g., for a control (e.g., positive and/or negative control) and a sample suspected of comprising an analyte. In some embodiments, a mean τₒₙ and/or τ_{off} is determined for each of a plurality of spots imaged on a solid support, e.g., for a control (e.g., positive and/or negative control) and a sample suspected of comprising a analyte. In some embodiments, a plot of τₒₙ versus τ_{off} (e.g., mean τₒₙ and τ_{off}, time-averaged τₒₙ and τ_{off}, etc.) for all imaged spots indicates the presence or absence of the analyte in the sample.

### Fluorescent Moieties

In some embodiments, a query probe comprises a fluorescent moiety (e.g., also known as a “fluorophore” or a “fluor”). A wide variety of fluorescent moieties is known in the art and methods are known for linking a fluorescent moiety to query probes.

Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4, 7, 2′, 7′-tetrachlorofluorescein (TET), 5- or 6-carboxy-4′5′2′4′5′7′ hexachlorofluorescein (HEX), 5′ or 6′-carboxy-4′,5′-dichloro-2, ‘7’-dimethoxyfluorescein (JOE), 5-carboxy-2′,4′,5′,7′-tetrachlorofluorescein (ZOE), rhodol, rhodamine, tetramethylrhodamine (TAMRA), 4,7-dlchlorotetramethyl rhodamine (DTAMRA), rhodamine X (ROX), and Texas Red. Examples of cyanine dyes that may find use with the present invention include but are not limited to Cy 3, Cy 3B, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

In some embodiments, the fluorescent moiety comprises a fluorescent protein (e.g., a green fluorescent protein (GFP), a modified derivative of GFP (e.g., a GFP comprising S65T, an enhanced GFP (e.g., comprising F64L)), or others known in the art such as, e.g., blue fluorescent protein (e.g., EBFP, EBFP2, Azurite, mKalama1), cyan fluorescent protein (e.g., ECFP, Cerulean, CyPet, mTurquoise2), and yellow fluorescent protein derivatives (e.g., YFP, Citrine, Venus, YPet). Embodiments provide that the fluorescent protein may be covalently or noncovalently bonded to one or more query probes, analytes, and/or capture probes.

Fluorescent dyes include, without limitation, d-Rhodamine acceptor dyes including Cy 5, dichloro[R110], dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′· Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz 2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl) anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof.

Several classes of fluorogenic dyes and specific compounds are known that are appropriate for particular embodiments of the technology: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin. In some embodiments the fluorescent moiety a dye that is xanthene, fluorescein, rhodamine, BODIPY, cyanine, coumarin, pyrene, phthalocyanine, phycobiliprotein, ALEXA FLUOR® 350, ALEXA FLUOR® 405, ALEXA FLUOR® 430, ALEXA FLUOR® 488, ALEXA FLUOR® 514, ALEXA FLUOR® 532, ALEXA FLUOR® 546, ALEXA FLUOR® 555, ALEXA FLUOR® 568, ALEXA FLUOR® 568, ALEXA FLUOR® 594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, or a squaraine dye. In some embodiments, the label is a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference in its entirety.

In some embodiments the label (e.g., a fluorescently detectable label) is one available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, all of which are incorporated herein by reference (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO740).

One of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

### Quencher Moieties

In some embodiments, a query probe comprises a quencher moiety. A wide variety of quencher moieties is known in the art and methods are known for linking a quencher moiety to a query probe. As used herein, “quenching group” or “quencher moiety” and similar terms refers to any fluorescence-modifying group that can attenuate, at least partly, the energy (e.g., light) emitted by a fluorescent moiety. This attenuation is referred to herein as “quenching”. Hence, irradiation of the fluorescent moiety in the presence of the quencher moiety leads to an emission signal from the fluorescent moiety that is less intense than expected, or even completely absent. Quenching typically occurs through energy transfer between the fluorescent moiety and the quencher moiety or by ground state stabilization wherein the quencher and fluorophore form a complex that prevents or inhibits excitation of the fluorescent moiety and thus minimizes and/or eliminates fluorescence by the fluorescent moiety.

Further, the technology is not limited in the type, structure, or composition of the quencher moiety. Exemplary quenching moieties include a Black Hole Quencher, an Iowa Black Quencher, and derivatives, modifications thereof, and related moieties. Exemplary quenching moieties include BHQ-0, BHQ-1, BHQ-2, and BHQ-3. Further examples of quenchers include colloidal nanocrystals (e.g., quantum dots and gold nanoparticles) that can quench fluorescence through energy transfer. In addition, in some embodiments, nonspecific interactions between fluorophores and nearby molecules (e.g., proteins, DNA duplexes) produce quenching (see, e.g., Hwang (2014) Chem Soc Rev 43:1221-29, incorporated herein by reference). In some embodiments, any molecule, moiety, or atom in the local environment of a fluorescent moiety that produces fluorescence quenching or enhancement (e.g., by modifying the molecular and/or electronic structure of the fluorescent moiety) finds use in the technology described herein.

In some embodiments, fluorescent moiety-quencher moiety include, e.g., DLO-FB1 (5′-FAM/3′-BHQ-1) DLO-TEB1 (5′-TET/3′-BHQ-1), DLO-JB1 (5′-JOE/3′-BHQ-1), DLO-1-HB1 (5′-HEX/3′-BHQ-1), DLO-C3B2 (5′-Cy3/3′-BHQ-2), DLO-TAB2 (5′-TAMRA/3′-BHQ-2), DLO-RB2 (5′-ROX/3′-BHQ-2), DLO-C5B3 (5′-Cy5/3′-BHQ-3), DLO-C55B3 (5′-Cy5.5/3′-BHQ-3), MBO-FB1 (5′-FAM/3′-BHQ-1), MBO-TEB1 (5′-TET/3′-BHQ-1), MBO-JB1 (5-JOE/3′-BHQ-1), MBO-HB1 (5′-HEX/3′-BHQ-1), MBO-C3B2 (5′-Cy3/3′-BHQ-2), MBO-TAB2 (5′-TAMRA/3′-BHQ-2), MBO-RB2 (5′-ROX/3′-BHQ-2); MBO-C5B3 (5′-Cy5/3′-BHQ-3), MBO-C55B3 (5′-Cy5.5/3′-BHQ-3) or similar FRET pairs available from Biosearch Technologies, Inc. of Novato, Calif. See, e.g., U.S. Pat. App. Pub. No. US20100317005 incorporated herein by reference.

### Methods

Some embodiments provide a method of identifying an analyte by repetitive query probe (e.g., aptamer probe) binding. In some embodiments, methods comprise immobilizing an analyte to a solid support. In some embodiments, the solid support is a surface (e.g., a substantially planar surface, a rounded surface), e.g., a surface in contact with a bulk solution, e.g., a bulk solution comprising analyte. In some embodiments, immobilizing an analyte to a solid support comprises stably binding the analyte to a capture probe that is immobilized to the solid support.

In some embodiments, the solid support is a freely diffusible solid support (e.g., a bead, a colloidal particle, e.g., a colloidal particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)), e.g., that freely diffuses within the bulk solution, e.g., a bulk solution comprising the analyte. In some embodiments, immobilizing an analyte to a solid support comprises covalent interaction between the solid support and analyte. In some embodiments, immobilizing an analyte to a solid support comprises non covalent interaction between the solid support and analyte. In some embodiments, the analyte (e.g., a molecule, e.g., a molecule such as, e.g., a protein, peptide, nucleic acid, small molecule, lipid, metabolite, drug, etc.) is stably immobilized to a surface and methods comprise repetitive (e.g., transient, low-affinity) binding of a query probe to the target analyte. In some embodiments, methods comprise detecting and/or recording the repetitive (e.g., transient, low-affinity) binding of a query probe to the target analyte.

In some embodiments, methods comprise generating a dataset (e.g., a series of time-resolved images (e.g., a movie or video) comprising a signal produced from query probe (e.g., aptamer probe) binding to the analyte (e.g., a dataset of query probe signal as a function of time). In some embodiments, methods comprise generating a dataset comprising a signal produced from query probe (e.g., aptamer probe) binding to the analyte (e.g., a dataset of query probe signal as a function of time) and information (e.g., coordinates, e.g., x, y coordinates) describing the spatial position on the surface of the query probe binding to the analyte. In some embodiments, the dataset is processed (e.g., manipulated, transformed, visualized, etc.), e.g., to improve the spatial resolution of the query probe binding events. For example, in particular embodiments, the dataset (e.g., comprising query probe signal as a function of time and information (e.g., coordinates, e.g., x, y coordinates) describing the spatial position on the surface of the query probe binding to the analyte) is subjected to processing. In some embodiments, the processing comprises a frame-by-frame comparison (e.g., subtraction) process to generate differential intensity profiles showing query probe binding or dissociation events (e.g., as identified by signal intensity changes) within each frame of the time series data. Data collected during the development of the technology described herein indicate that the differential intensity profiles have a higher resolution than the query probe binding signal vs. position map. In some embodiments, after determining the spatial position (e.g., x, y coordinates) of each query probe binding and/or dissociation event, a plurality of events is clustered according to spatial position and the kinetics of the events within each cluster are subjected to statistical analysis to determine whether the cluster of events originates from a given target analyte.

For instance, some embodiments of methods for quantifying one or more surface-immobilized or diffusing target analytes comprise one or more steps including, e.g., measuring the signal of one or more transiently binding query probes to the immobilized target analyte(s) with single molecule sensitivity. In some embodiments, methods comprise tracking (e.g., detecting and/or recording the position of) target analytes independently from query probe binding. In some embodiments, the methods further comprise calculating the time-dependent probe binding signal intensity changes at the surface. In some embodiments, the methods further comprise calculating the time-dependent probe binding signal intensity changes at the surface as a function of position (e.g., x, y position). In some embodiments, calculating the time-dependent query probe binding signal intensity changes at the surface (e.g., as a function of position (e.g., x, y position)) produces a “differential intensity profile” for query probe binding to the analyte. In some embodiments, the methods comprise determining the position (e.g., x, y position) of each query probe binding and dissociation event (“event”) (e.g., with sub; pixel accuracy) from a differential intensity profile. In some embodiments, methods comprise grouping events into local clusters by position (e.g., x, y position) on the surface, e.g., to associate events for a single immobilized target analyte. In some embodiments, the methods comprise calculating kinetic parameters from each local cluster of events to determine whether the cluster originates from a particular analyte, e.g., from transient probe binding to a particular analyte.

Embodiments of methods are not limited in the analyte that is detected. For example, in some embodiments the analyte is polypeptide, e.g., a protein or a peptide. In some embodiments, the target analyte is a nucleic acid. In some embodiments, the target analyte is a small molecule.

In some embodiments, the interaction between the target analyte and the query probe is distinguishably influenced by a covalent modification of the target analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, the analyte is a nucleic acid comprising a covalent modification to a nucleobase, a ribose, or a deoxyribose moiety of the target analyte.

In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid. In some embodiments, the query probe is a nucleic acid aptamer. In some embodiments, the aptamer is optimized (e.g., in length and/or base composition) as described herein.

In some embodiments, the query probe is a low affinity antibody, antibody fragment, or nanobody.

In some embodiments, the query probe is a DNA-binding protein, RNA-binding protein, or a DNA-binding ribonucleoprotein complex.

In some embodiments, the position, e.g., the (x,y) position, of each binding or dissociation event is determined by subjecting the differential intensity profile to centroid determination, least squares fitting to a Gaussian function, least square fitting to an airy disk function, least-squares fitting to a polynomial function (e.g., a parabola), or maximum likelihood estimation.

In some embodiments, the capture probe is a high affinity antibody, antibody fragment, or nanobody. In some embodiments, the capture probe is a nucleic acid. In some embodiments, the capture probe is an aptamer. In some embodiments, capture is mediated by a covalent bond cross-linking the target analyte to the surface. In some embodiments, the target analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

### Aptamer Probes

Sensitive and accurate detection of analytes is important for clinical diagnostics. In some embodiments, the technology provides for the sensitive and specific detection of analytes utilizing aptamer detection probes (e.g., aptamers comprising a fluorescent moiety). In some embodiments, the aptamer comprises a nucleic acid. In some embodiments, the aptamer comprises DNA. In some embodiments, the aptamer comprises RNA. In some embodiments, the aptamer comprises RNA comprising non-natural and/or modified bases.

In some embodiments, the technology comprises capturing an analyte from a sample (e.g., biological sample) onto a solid support (e.g., a glass or a quartz surface; a diffusible solid support such as a bead) comprising a capture probe (e.g., a primary antibody) to provide a captured analyte; and detecting the captured analyte using a transiently binding aptamer (e.g., an aptamer comprising a fluorescent moiety). In some embodiments, the technology comprises capturing an analyte from a sample (e.g., biological sample) onto a solid support (e.g., a glass or a quartz surface; a diffusible solid support such as a bead) comprising a capture probe (e.g., an oligonucleotide) to provide a captured analyte; and detecting the captured analyte using a transiently binding aptamer (e.g., an aptamer comprising a fluorescent moiety). In some embodiments, the technology comprises capturing an analyte from a sample (e.g., biological sample) onto a solid support (e.g., a glass or a quartz surface; a diffusible solid support such as a bead) comprising a capture probe (e.g., an aptamer) to provide a captured analyte; and detecting the captured analyte using a transiently binding aptamer (e.g., an aptamer comprising a fluorescent moiety).

In some embodiments, the transient binding of the aptamer (e.g., an aptamer comprising a fluorescent moiety) to the analyte is monitored using single molecule microscopy and/or digital single molecule counting to generate a time dependent fluorescence intensity signal (a “kinetic fingerprint”) that is used to detect, quantify, identify, or otherwise characterize the analyte (e.g., to describe the presence, absence, amount, concentration, and/or state of the analyte).

In some embodiments, the aptamer probe technology finds use in detecting, quantifying, identifying, or characterizing a protein analyte. In some embodiments, the technology uses transient binding of an aptamer query probe (e.g., a low-affinity, high-specificity aptamer query probe) to a protein analyte to generate a time-dependent fluorescence intensity signal (a “kinetic fingerprint”) for detecting, quantifying, identifying, or characterizing a protein analyte.

Detecting analytes (e.g., proteins) with aptamer query probes as described herein provides several advantages over some other conventional detection technologies, including some that use antibodies. Aptamers are non toxic and non-immunogenic; aptamers can be chemically synthesized easily; and aptamers are more stable and easily modifiable than antibodies. Furthermore, aptamers can be designed in silico, synthesized using solid phase chemical synthesis of DNA and RNA, and labeled with site-specific and stoichiometric fluorophore labels that do not affect or minimally affect the analyte binding site. Accordingly, embodiments of the present technology overcome some of the shortcomings of conventional technologies and provides highly sensitive and specific detection of analytes, e.g., protein biomarkers.

In particular, experiments conducted during the development of embodiments of the technology described herein indicated the applicability of fluorophore-labelled aptamers as rationally tunable probes for the highly sensitive and specific detection of two clinically relevant protein biomarkers, VEGF₁₆₅ and IL-8, by SiMREPS. As with previously reported protein-SiMREPS assays using Fab antibody detection probes, aptamer based SiMREPS achieves low-femtomolar (sub-pg/mL) LODs in animal and human serum, exceeding the sensitivity of commercial ELISAs by more than two orders of magnitude, and can be performed using a wash free protocol that bypasses the tedious multi-step washing protocols required of most immunoassays. Accordingly, in some embodiments, the technology detects an analyte present in a sample at a concentration of 1 to 10 fM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 fM). In some embodiments, the technology detects an analyte present in a sample at a concentration of 0.01 to 1 pg/mL (e.g., 0.01, 0.02, 0.05, 0.1, 0.2, 0.5, or 1 pg/mL).

Further, aptamers provide several advantages as SiMREPS probes compared to antibodies, including the ability to rationally tune their kinetics of interaction with the target through selective mutation or truncation, and the ease of introducing site-specific and stoichiometric fluorophore modifications using chemical synthesis. These features have direct impact on the use of aptamers as SiMREPS probes by reducing the required data acquisition time (faster kinetics result in faster data collection) and reducing the complexity of intensity versus time traces (two-state intensity behavior is simpler to analyze than multi-state behavior). Rationally designed aptamers may be synthesized at low cost using relatively simple equipment. Aptamers comprising nuclease-resistant nucleotides are tolerant of varied storage conditions. Importantly, the aptamers examined in this study showed less nonspecific binding to assay surfaces and matrix proteins than most antibody probes examined previously.

Data were collected during experiments testing two kinds of rational modification of aptamer sequences to improve their performance as SIMREPS probes: 1) single-nucleotide changes were introduced into the conserved target-binding region to reduce the affinity of tightly binding probes; and 2) sequence not directly involved in the aptamer analyte interaction was removed (e.g., by truncation) to increase association kinetics.

Importantly, prototyping and testing aptamers involved a small number of trials (e.g., <10) to optimize the aptamer design. Aptamers suitable to rapid rational redesign are available from SELEX approaches against over 1,000 human proteins (Brody (2012” “Life's simple measures: unlocking the proteome” J Mol Biol 422(5): 595-606; Gold (2010) “Aptamer-Based Multiplexed Proteomic Technology for Biomarker Discovery” Plos One 5(12); Hathout (2015) “Large-scale serum protein biomarker discovery in Duchenne muscular dystrophy” PNAS 112(23): 7153-58; Thanasupawat (2021) “Slow Off-Rate Modified Aptamer (SOMAmer) Proteomic Analysis of Patient-Derived Malignant Glioma Identifies Distinct Cellular Proteomes” Int J Mol Sci 22(17), each of which is incorporated herein by reference); thus, embodiments of the technology described herein are readily adaptable to detect, quantify, identify, and/or characterize many biomarkers of disease. In addition, as with antibody probes, binding and/or dissociation rates can be increased by elevating the temperature or by manipulating the ionic strength of the solution, resulting in improved assay performance or reduced measurement time (see, e.g., U.S. Pat. App. Pub. No. 2021/0292837, incorporated herein by reference). Thus, although antibody detection probes for SIMREPS are readily generated by phage display, the addition of aptamers as detection probes provides more paths to generating probes with the desired rapid binding and dissociation kinetics, and therefore is expected to drastically facilitate the development of high-performance assays against a wide range of target analytes important in research and diagnostics.

### EXAMPLES

### Aptamer Probes

During the development of embodiments of the technology described herein, SiMREPS assays were performed using aptamer query probes. During the development of embodiments of the technology described herein, experiments were conducted using aptamers as detection probes in SiMREPS to detect two clinically relevant biomarkers, VEGF₁₆₅ and IL-8. The experiments used a wash free protocol and had a limit of detection in the low femtomolar range (3-9 fM). Data collected during these experiments indicated that the kinetics of RNA aptamers can be rationally optimized for use as SiMREPS query probes by mutating one or more nucleotides in the conserved binding region or by modifying the length or sequence of the aptamer nucleic acid. Data collected indicated that the technology detected endogenous IL-8 from human serum at a concentration below the detection limit of commercial ELISAs.

### Materials and Methods

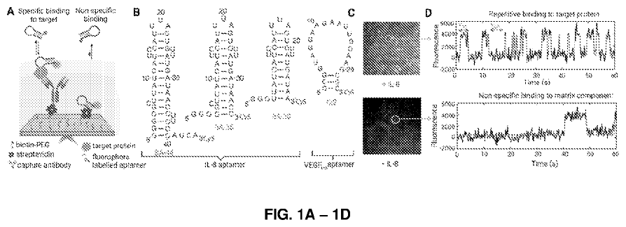

Oligonucleotides and proteins—All 2′-fluoropyrimidine modified and 3′-Cy5-labelled RNA oligonucleotides were purchased from Integrated DNA Technologies (IDT, www.idtdna.com) with high-performance liquid chromatography (HPLC) purification. Oligonucleotide sequences are shown in FIG. 1B and Table 1.

TABLE 1
RNA Aptamers

| Aptamer | | SEQ |
|---|---|---|
| name | Aptamer sequences with nucleobase modifications | ID NO: |
| 8A-44 | 5′GGGGG/i2FC//i2FU//i2FU/A/i2FU//i2FC/A/i2FU// | 1 |
| | i2FU//i2FC//i2FC/A/i2FU//i2FU//i2FU/AG/i2FU/G/ | |
| | i2FU//i2FU/A/i2FU/GA/i2FU/AA/i2FC//i2FC//i2FU/ | |
| | CUCCCAUA 3′Cy5 | |
| 8A.35 | 5′GGGGG/i2FC//i2FU//i2FU/A/i2FU//i2FC/A/i2FU// | 2 |
| | i2FU//i2FC//i2FC/A/i2FU//i2FU//i2FU/AG/i2FU/G/i2FU/ | |
| | /i2FU/A/i2FU/GA/i2FU/AA/i2FC//i2FC//3′Cy5 | |
| 8A-30 | 5′Cy5GGG/i2FU//i2FU/A/i2FU//i2FC/A/i2FU//i2FU// | 3 |
| | i2FC//i2FC/A/i2FU//i2FU//i2FU/AG/i2FU/G/i2FU//i2FU/ | |
| | A/i2FU/GA/i2FU/AA 3′ | |
| t22 | 5′G/i2FC/GG/i2FU/AGGAAGAA/i2FU//i2FU/GGAAG/ | 4 |
| | i2FC/G/i2FC//3′Cy5 | |

In Table 1, “i2FC” indicates a fluoro C (2′-fluoro-cytodine), “i2FU” indicates a fluoro-U (2′-fluoro-uridine), “5′Cy5” indicates a Cy5 label at the 5′ end, and “3′ Cy5” indicates a Cy5 label at the 3′ end. Oligonucleotides were suspended in TE buffer (10 mM Tris, 0.1 mM EDTA, pH 8.0) to a final concentration of 100 μM and aliquoted and stored at −80° C. Recombinant human IL-8 (Ser28-Ser99, catalog number BR-1098) was provided in lyophilized form by Bio-Rad Laboratories, Inc. Biotinylated recombinant human IL-8 (catalogue number B-CXCL8-2 μg) was purchased from Chemotactics in lyophilized form. Recombinant human VEGF₁₆₅ was purchased from Abcam (catalog number ab9571) as a lyophilized powder. Antigens were suspended in 1×PBS, pH 7.4 (Gibco) supplemented with 10 mg/mL BSA (Thermo Scientific Blocker BSA (10×) in PBS; catalog number 37525) as a carrier, aliquoted, and frozen at −80° C. Monoclonal IL-8 capture antibody (catalog number EPR19358-108) and monoclonal VEGF₁₆₅ capture antibody (IgG) were purchased from Abcam and Bio-Rad Laboratories, respectively. The VEGF₁₆₅ capture antibody was provided as lyophilized powder from a 0.2 μm-filtered solution in PBS with 5% trehalose. Both of the capture antibodies were free of BSA and azide to facilitate labelling with biotin NHS ester. Horse serum (catalogue number H1270) and human serum (catalogue number H4522) were purchased from Sigma Aldrich.

Biotinylation of capture antibody—Monoclonal capture antibodies were biotinylated by amine-NHS ester coupling using biotin N-hydroxysuccinimidyl ester (Sigma Aldrich, Catalog number II1759-100) in reactions containing a molar biotin antibody ratio of 5:1 in 1×PBS, pH 7.4 and reacted at room temperature for 1 hour. Biotin IgG conjugates were purified using Zeba Spin desalting columns (ThermoFisher, Catalog number 89882, 7K MWCO) according to the manufacturer recommended protocol, followed by overnight dialysis at 4° C. (Slide-A-Lyzer Dialysis Cassette, ThermoFisher, 3.5K MWCO) against 1×PBS, pH 7.4. The fraction of biotinylated IgG was estimated by electrophoretic mobility shift assay in the presence or absence of excess streptavidin and ranged from 70% to 80%. Capture antibodies were aliquoted and frozen at −80° C.

Preparation of slide surfaces for single-molecule microscopy—Glass coverslips (No. 1.5, 24×50 mm, VWR catalog number 48393-241) were functionalized with a 1:100 mixture of biotin-PEG-SVA and mPEG-SVA (Laysan Bio, Inc., catalog numbers MPEG-SVA-5000-1g and BIO-PEG-SVA-5K-100 MG) as previously described by Chatterjee (2020) “Direct kinetic fingerprinting and digital counting of single protein molecules” PNAS 117(37): 22815-22, incorporated herein by reference. Coverslips were stored under aluminum foil in a nitrogen purged cabinet until use (up to 4 weeks). Prior to an experiment, 2 to 6 sample cells were attached to each coverslip by cutting an approximately 2-cm length from the wider end of micropipette tips (Thermo Fisher, catalog number 02-682-261), discarding the narrower segment of the pipet tip, placing the wide end down on the PEGylated glass coverslip, and sealing the edges with epoxy adhesive (Ellsworth Adhesives, catalog number 4001).

TIRF microscopy—SiMREPS experiments were performed using Olympus IX-81 objective-type TIRF microscopes equipped with cellTIRF and z-drift control modules (ASI CRISP). Cy5-labelled detection aptamers were excited in TIRF mode with a theoretical penetration depth of approximately 80 nm using a fiber-coupled diode laser (OBIS 637 nm LX, 100 mW) with an incident light intensity of approximately 100 W/cm², and fluorescence emission was detected using an EMCCD (Photometrics Evolve) with an exposure time of 200 or 500 ms, after passing through a dichroic mirror and emission filter (ET655LP-TRF). In some experiments, an objective heater (Bioptechs) was used to raise the observation temperature to as high as 29° C. (calibrated against a reference thermistor provided by the manufacturer for the specific sample cell geometry used in this study).

Imaging solution—Unless otherwise specified, all SiMREPS assays were carried out in an imaging solution comprising 1×PBS, pH 7.4 or 6×PBS, pH 7.4 (Gibco); an oxygen scavenger system (see, e.g., Aitken (2008) “An oxygen scavenging system for improvement of dye stability in single molecule fluorescence experiments” Biophys J 94(5): 1826-35, incorporated herein by reference) comprising 5 mM 3,4-dihydroxybenzoic acid (Fisher, catalog number AC114891000), 0.05 mg/mL protocatechuate 3,4-dioxygenase (Sigma Aldrich, catalog number P8279-25UN), and 1 mM Trolox (Fisher, catalog number 218940050); and 75-100 nM fluorophore labelled detection aptamers.

SiMREPS assays of recombinant antigens-All sample handling was performed in GeneMate low-adhesion 1.7-mL microcentrifuge tubes, and dilutions were performed in 25% horse serum, 0.75×PBS, pH 7.4, and 7.5 mg/mL BSA. The slide surface was washed with 100 μL of T50 buffer (10 mM Tris-HCl, 50 mM NaCl, 1 mm EDTA, pH 8.0) for 10 minutes followed by the addition of 40 μL 1 mg/mL streptavidin. After 10 minutes, excess streptavidin was removed, and the sample chamber washed three times with 100 μL of 1×PBS. Next, the coverslip was coated with the biotinylated capture antibody by adding 40 μL of a solution containing 10 nM of biotinylated capture antibody in 1×PBS buffer and incubating for 30 minutes. Excess antibody was removed and the sample wells washed three times with 100 μL of 1×PBS. A 100-μL portion of the antigen or blank solution was added to the sample chamber and incubated for 1 hour to capture the antigen on the coverslip surface. The sample was removed, the sample cell washed twice with 100 μL of 1×PBS, and 200 μL of imaging solution added. Kinetic fingerprints of aptamer binding were immediately imaged by TIRF microscopy using an acquisition time of 1 to 2 minutes per field of view (FOV) for IL-8 or 10 min per FOV for VEGF₁₆₅.

Wash-free SiMREPS standard curves-Capture antibody-coated glass coverslips were prepared as described above. A 200-μL volume of a mixture containing varying concentrations of recombinant human IL-8 and VEGF₁₆₅ spiked into 1% horse serum in 1×PBS (IL-8 standard curve) or 6×PBS (VEGF₁₆₅ standard curve), pH 7.4, containing 5 mM 3,4-dihydroxybenzoic acid, 0.05 mg/mL protocatechuate 3,4-dioxygenase, 1 mM Trolox, and 75 nM (IL-8 detection) or 100 nM (VEGF₁₆₅ detection) of fluorophore-labelled aptamer was added to the sample well and incubated for 40 minutes. Kinetic fingerprints of detection aptamer binding were then immediately imaged by TIRF microscopy using an acquisition time of 1 minute per FOV at 24° C. for IL-8 detection and 5 minutes per FOV at 21° C. for VEGF₁₆₅ detection.

Wash-free measurement of endogenous IL-8 in human serum—Capture antibody coated coverslips were prepared as described above. Samples of 2% and 10% human serum were prepared by adding 4 μL or 20 μL of human serum to a total volume of 200 μL imaging solution containing: 1×PBS, pH 7.4, 5 mM 3,4-dihydroxybenzoic acid, 0.05 mg/mL protocatechuate 3,4-dioxygenase, 1 mM Trolox, and 75 nM of detection aptamer. This 200 μL mixture was added to the sample well and incubated for 40 minutes. Kinetic fingerprints of detection aptamer binding were then immediately imaged by TIRF microscopy using an acquisition time of 1 minute per FOV at an acquisition temperature of 24° C.

ELISA standard curves—Standard curves for detection of IL-8 from 100% human serum were performed using an IL-8 sandwich ELISA kit (Abcam, catalog number ab214030) according to the manufacturer recommended protocol. The manufacturer claimed LOD for the ELISA kit is 211 fM (1.8 pg/mL).

Analysis of SiMREPS data—SiMREPS data were analyzed using custom MATLAB code to identify sites of fluorophore labelled aptamer probe binding and to analyze the kinetics of repeated binding as described previously using a diffraction-limited analysis pipeline (see, e.g., Johnson Buck (2019) “A guide to nucleic acid detection by single-molecule kinetic fingerprinting” Methods 153:3-12, incorporated herein by reference). Briefly, regions of repeated probe binding and dissociation (regions of interest, ROIs) in the FOV were identified by determining the average absolute frame-to-frame change in intensity at each pixel to create an intensity fluctuation map and then defining ROIs as the 3×3-pixel regions centered on local maxima within the fluctuation map. Next, the integrated, background subtracted intensity within each ROI was calculated for each frame in the movie to generate an intensity versus time trace. These candidate traces were subjected to hidden Markov modeling (HMM) using a version of vbFRET (see, e.g., Bronson (2009) “Learning Rates and States from Biophysical Time Series: A Bayesian Approach to Model Selection and Single-Molecule FRET Data” Biophys J 97(12): 3196-3205, incorporated herein by reference). The idealized trace generated by HMM was used to determine several parameters for SIMREPS kinetic fingerprinting analysis: N_{b+d}, the number of binding and dissociation events; τ_{on,median} and τ_{off,median}, the median dwell times in the probe-bound and probe-unbound states, respectively; τ_{off,max}, the maximum dwell time in the probe-unbound state; and S/n, the signal-to-noise ratio, defined as the standard deviation of the fluorescence intensity divided by the mean intensity difference between bound and unbound states. Threshold values for each of these parameters to count a trace as a positive detection event were optimized heuristically for each probe antigen pair.

### Results

Aptamer based kinetic fingerprinting assay design-During the experiments described herein, the aptamer-based SiMREPS assays use two probes: 1) a capture probe (e.g., a capture antibody (e.g., an IgG)) that stably binds to an analyte (e.g., a protein) and thus has very slow dissociation kinetics or that does not dissociate on the time scale of the experiment; and 2) a low-affinity RNA aptamer having rapid dissociation kinetics with respect to the analyte (k_{off} approximately 0.05 to 0.5 s⁻¹) (See, e.g., FIG. 1A). The capture probe is modified with biotin to provide surface immobilization of the target analyte (e.g., protein) via a streptavidin bridge to a biotin-PEG (polyethylene glycol)-coated coverslip, while the aptamer is modified with an organic fluorophore (e.g., at the 3′ and/or 5′ end) to provide detection of its binding to the coverslip surface by total internal reflection fluorescence (TIRF) microscopy (FIG. 1A and FIG. 1C). The aptamers comprise 2′-fluoro modifications at all pyrimidine nucleotides to increase the stability of the aptamers in serum (see, e.g., Ruckman (1998) “2′-fluoropyrimidine RNA-based aptamers to the 165-amino acid form of vascular endothelial growth factor (VEGF (165))—Inhibition of receptor binding and VEGF-induced vascular permeability through interactions requiring the exon 7-encoded domain” J Biol Chem 273(32): 20556-67; Sung (2014) “Inhibition of human neutrophil activity by an RNA aptamer bound to interleukin-8.” Biomaterials 35(1): 578-89, incorporated herein by reference). As in immunoassays, the fluorophore-labelled aptamers exhibited some nonspecific binding to assay surfaces, albeit at much lower levels than most fluorophore-labelled detection antibodies, upon single molecule observation (FIG. 1C). In the absence of kinetic fingerprinting, this nonspecific binding would constitute background signal that could not be distinguished from specific binding. However, the nonspecific binding typically exhibits kinetics of interaction quite distinct from the more repetitive specific binding of the detection aptamer to the target protein, making it readily distinguishable by analysis of localized fluorescence intensity fluctuations over time (FIG. 1D). Most notably, nonspecific interactions occur with much lower frequency at any given location on the surface, while the specific binding to the target protein generates repetitive binding at the same location (FIGS. 1C and 1D). Thus, by applying empirically determined thresholds that include a minimum number of binding (fluorescence-on transition) and dissociation (fluorescence-off transition) events (N_{b+d}) and minimum and maximum values of the median dwell time in the probe-bound state (ι_{on,median}), distinguish single molecule traces arising from specific binding can be distinguished from signals produces from non-specific binding. Kinetic filtering removes false positive counts and retains the majority of true positives, demonstrating the high specificity of aptamer based single molecule protein detection by kinetic fingerprinting. Significantly, in contrast to the multi-state intensity traces observed with inherently non-stoichiometrically labelled antibodies, the single molecule time traces collected during the experiments described herein show two state behavior since each copy of aptamer has (at most) one copy of active fluorophore (FIG. 1D). This simplifies the HMM fitting of the intensity versus-time data and hence permits extraction of more accurate kinetic information for the digital counting of true positives.

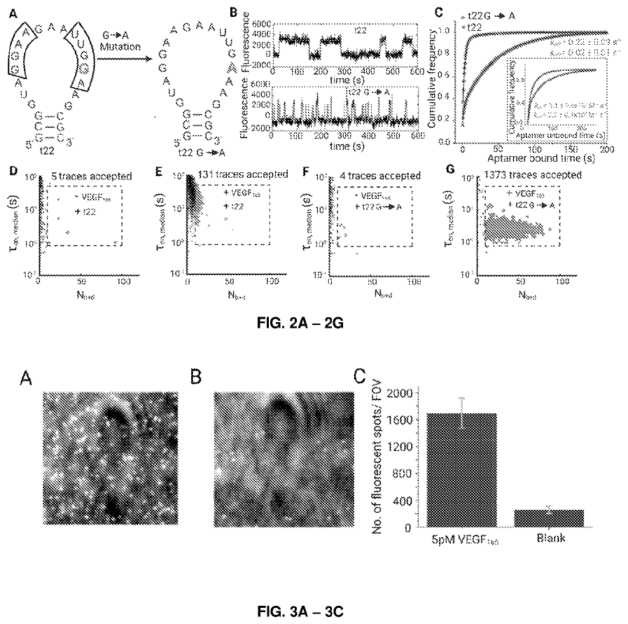

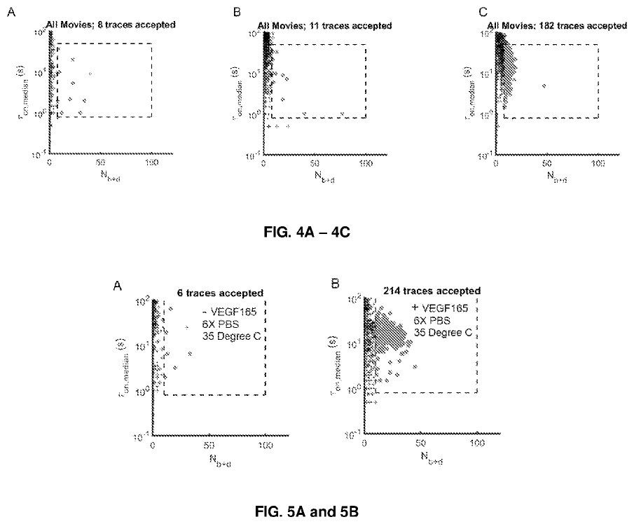

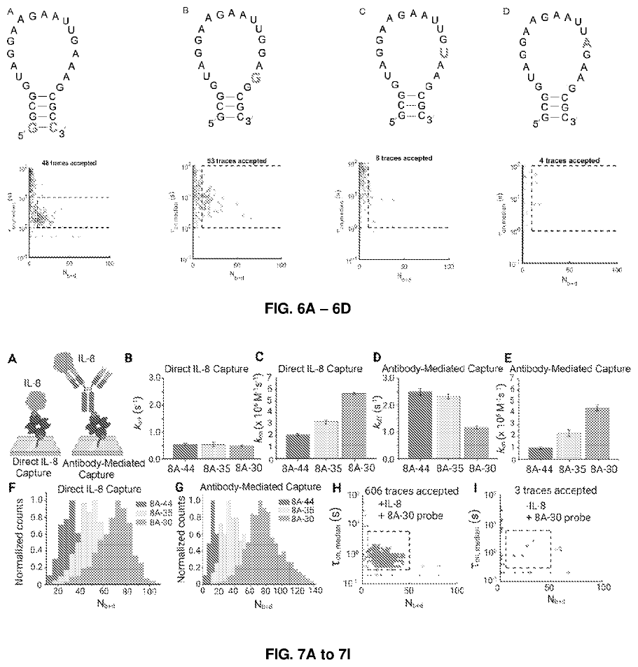

Aptamer modification and assay optimization for VEGF₁₆₅ detection—For the detection of VEGF₁₆₅, the aptamer t22 was adapted from the literature (see, e.g., Ruckman (1998) 2′-fluoropyrimidine RNA-based aptamers to the 165-amino acid form of vascular endothelial growth factor (VEGF(165))—Inhibition of receptor binding and VEGF-induced vascular permeability through interactions requiring the exon 7-encoded domain” J Biol Chem 273(32): 20556-67, incorporated herein by reference). (FIGS. 1B and 2A). The aptamer was generated by SELEX from a randomized library of RNA sequences to bind VEGF₁₆₅ with high affinity and high specificity. Consistent with the expected specific binding of the aptamer to VEGF₁₆₅, single-molecule fluorescence imaging revealed 8 to 10 times more fluorescent spots per FOV in the sample well containing VEGF₁₆₅ than in a control well without VEGF₁₆₅ (FIG. 3A-3C). However, consistent with the reported slow dissociation rate (k_{d} of approximately 0.012±0.0025 s⁻¹), stable binding of t22 to the surface captured VEGF₁₆₅ was observed in single molecule imaging, with lifetimes of several tens of seconds in the bound state, making it difficult to distinguish true and false positives on the basis of repeated binding (FIG. 4A-4C). However, after increasing the salt concentration 6-fold (from 1×PBS to 6×PBS), a subpopulation of traces in the sample well showed kinetics distinct from the blank well and could thus be identified as true positives by applying appropriate kinetic filtering parameters (FIG. 2D, FIG. 2E, and FIG. 4A-C). This influence of ionic strength indicates the involvement of ionic interactions between the probe and antigen. Raising the temperature yielded further increases in kinetics but was not sufficient to separate the signal from the background completely (FIG. 5A and FIG. 5B; FIG. 2D and FIG. 2E). To further improve performance, five rationally designed mutations of 122 were evaluated to determine their impact on kinetics (FIG. 2A; FIG. 6A to 6D). Evaluating these mutations indicated that a single G-to-A mutation in a conserved region of the t22 sequence (t22 G→A) yielded the greatest improvement in performance (FIGS. 2A and 2B) due to its approximately 10-fold faster dissociation (k_{off}=0.22 s⁻¹ for t22 G→A vs k_{off}=0.02 s⁻¹ for t22) and approximately 2.3-fold faster association (kₒₙ=5.1×10⁵ M⁻¹s⁻¹ for t22 G→A vs kₒₙ=2.2×10⁵ M⁻¹s⁻¹ for t22) compared to the original t22 aptamer under identical buffer and imaging conditions (FIG. 2C). The increases in both dissociation and association rate accelerated the repeated binding of individual target molecules (FIG. 2B), increasing N_{b+d} and thus permitting substantially better resolution of specific from nonspecific kinetic behavior (FIGS. 2F and 2G). Thus, unlike with antibody probes, it is feasible to design site-specific nucleobase modifications by chemical synthesis to convert a stably binding aptamer to a lower-affinity SiMREPS probe showing rapid, repeated binding and dissociation.

Aptamer sourcing and assay optimization for IL-8 detection—The aptamers used for IL-8 detection are shown in FIG. 1B. The parent aptamer 8A-W (91 nt) was generated previously by the SELEX method against human IL-8 and was then truncated to generate 8A-44 (44 nt, 14.6 kDa), 8A-35 (35 nt, 11.8 kDa), and 8A-30 (30 nt, 10.1 kDa), without compromising binding to IL-8 (see, e.g., Sung (2014) “Inhibition of human neutrophil activity by an RNA aptamer bound to interleukin-8” Biomaterials 35(1): 578-89, incorporated herein by reference). Without being bound by theory, it was hypothesized that the more truncated versions of these aptamers might perform better as SiMREPS probes because smaller probes would have better access to the binding regions on analytes, especially when a small protein is immobilized by a capture antibody (see, e.g., Dey (2005) “An aptamer that neutralizes R5 strains of human immunodeficiency virus type 1 blocks gp 120-CCR5 interaction” J Virol 79(21): 13806-10; Lee (2006) “Aptamer therapeutics advance” Curr Opin Chem Biol 10(3): 282-89, each of which is incorporated herein by reference).

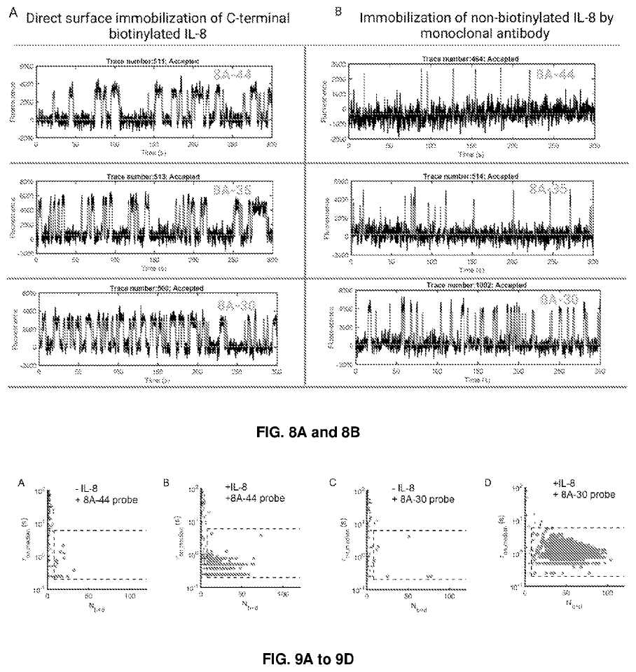

To systematically assess the effect of aptamer size on the kinetics of binding to the small (8.4 kDa) protein IL-8, two different antigen capture strategies were used: direct capture, in which C-terminally biotin labelled IL-8 was immobilized directly on the surface by the biotin-streptavidin interaction; and antibody mediated capture, in which non-biotinylated IL-8 was captured on the surface by a surface-immobilized biotinylated monoclonal antibody (FIG. 7A). Kinetic time trajectories of single IL-8 molecules interacting with the various Cy5-labelled aptamers show that binding and dissociation kinetics are systematically influenced by both aptamer truncation and the capture strategy (FIGS. 8A and 8B). To gain more quantitative insight, association and dissociation rate constants were calculated from the dwell time distributions in the aptamer-unbound and -bound states, respectively, and are shown in Table 2. In particular, Table 2 shows the binding parameters arising from interaction between Cy5-labelled 8A aptamers (at 75 nM) and IL-8 in 25% horse serum at 21° C. as determined by single-molecule fluorescence microscopy.

TABLE 2
8A aptamer binding parameters

| Capture | | kₒₙ × 10⁻⁶ | k_{off} | |
|---|---|---|---|---|
| strategy | Aptamer | (M⁻¹s⁻¹) | (s⁻¹) | K_{D}(μM) |
| Direct IL-8 | 8A-44 | 2.08 ± 0.12 | 0.55 ± 0.04 | 0.264 ± 0.061 |
| capture | 8A-35 | 3.21 ± 0.18 | 0.54 ± 0.09 | 0.168 ± 0.032 |
| | 8A-30 | 5.68 ± 0.09 | 0.50 ± 0.04 | 0.088 ± 0.010 |
| Antibody | 8A-44 | 0.99 ± 0.11 | 2.50 ± 0.13 | 2.52 ± 0.074 |
| mediated | 8A-35 | 2.29 ± 0.32 | 2.32 ± 0.09 | 1.01 ± 0.089 |
| capture | 8A-30 | 4.48 ± 0.28 | 1.17 ± 0.05 | 0.261 ± 0.018 |

In direct capture, all the aptamer variants show similar dissociation rates (k_{off} approximately 0.5 s⁻¹) (FIG. 7B), but the association rates decrease from 5.68×10⁶ M⁻¹s⁻¹ to 2.08×10⁶ M⁻¹s⁻¹ as the aptamer size increases (FIG. 7C). Without being bound by theory, it is contemplated that this inverse relationship between size and binding rate may result in part from the slower diffusion coefficient D of the probe with increasing molecular weight M, however, the expected relationship

$$D ∝ \frac{1}{\sqrt{M}}$$

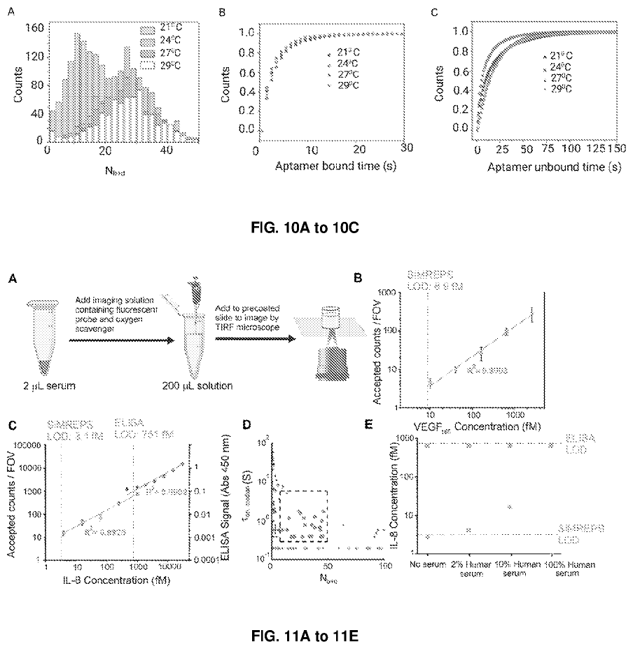

(see, e.g., Grushka and Kikta (1976) “Diffusion in Liquids .2. Dependence of Diffusion-Coefficients on Molecular-Weight and on Temperature” J Am Chem Soc 98(3): 643-48, incorporated herein by reference) would only account for an approximately 1.2-fold change in binding kinetics, indicating that other factors such as reduced productivity of collisions may play a role. In antibody-mediated capture, the dissociation rate from the IL-8 binding site increases for all the aptamers (FIG. 7D). The dissociation rate is fastest for 8A-44 (k_{off} approximately 2.5 s⁻¹) and slowest for 8A-30 (k_{off} approximately 1.17 s⁻¹), indicating that steric hindrance between the antibody and larger aptamers may be accelerating dissociation. Furthermore, antibody mediated capture reduces the association rates of all the aptamers, but by the greatest factor for the larger aptamers 8A-44 and 8A-35. For the short 8A-30 aptamer, the association rate only decreases by a factor of approximately 1.2 (FIG. 7E). The fast binding and dissociation of 8A-30 results in a higher number of binding/dissociation events (N_{b+d}) within a given observation period and, hence, better separation between signal and background kinetics, than 8A-35 or 8A-44 (FIG. 7F and FIG. 7G; FIG. 9A to FIG. 9D). This shows that the performance of an aptamer in SiMREPS can be improved by truncating non-conserved portions of the sequence, resulting in faster kinetics and therefore more rapid data acquisition and/or higher analytical performance. Such optimizations are difficult or impossible for antibody probes, which have a relatively fixed overall structure that cannot be easily modified. Due to its fast kinetics, 8A-30 was chosen for further use and optimization in SIMREPS assays of IL-8 in serum (FIG. 7H and FIG. 7I). As an additional optimization, slightly raising temperature from 21° C. to 24° C. further increased the kinetics of 8A-30, permitting a data acquisition time of only 1 minute per field of view without compromising signal/background discrimination (FIG. 7H and FIG. 7I; FIG. 10A to 10C). Further increasing to 27-29° C. did not result in significant improvements relative to 24° C.

Analytical performance and detection of endogenous IL-8 in human serum-During the development of embodiments of the technology described herein, experiments were conducted to investigate the sensitivity and specificity of aptamer-based SiMREPS protein assays. In particular, human VEGF₁₆₅ and IL-8 were spiked into horse serum at varying concentrations and then measured by SiMREPS using nuclease-resistant 2′-fluoropyrimidine modified RNA aptamers. The assays were performed using a wash free protocol wherein serum samples comprising spiked in protein were mixed with imaging buffer, added to a capture antibody-coated coverslip surface, and then imaged after a suitable incubation period (FIG. 11A). Hence, unlike in conventional assays, neither the sample nor the excess detection probe need to be washed away after sample addition. The acquisition parameters and kinetic filtering criteria for the quantification of VEGF₁₆₅ and IL-8 are shown in Table 3.

TABLE 3
Acquisition parameters and kinetic filtering criteria

| | IL-8 | VEGF₁₆₅ |
|---|---|---|
| Detection probe | Aptamer | Aptamer |
| | 8A-30 | t22 G→A |
| Detection probe | 75 | 100 |
| concentration (nM) | | |
| Exposure time per | 0.2 | 0.5 |
| frame (s) | | |
| Acquisition time | 1 | 5 |
| (minutes) | | |
| Acquisition temperature | 24 | 21 |
| (° C.) | | |
| Minimum r_{s/n} per event | 2 | 2 |
| Minimum r_{s/n} per trace | 2 | 3 |
| Minimum N_{b+d} | 8 | 5 |
| Maximum N_{b+d} | 50 | 100 |
| Minimum τ_{on, median} (s) | 0.25 | 1 |
| Maximum τ_{on, median} (s) | 2 | 5.5 |
| Minimum τ_{off, median} (s) | 0.8 | 2 |
| Maximum τ_{off, median} (s) | 50 | 200 |

As shown in FIG. 11B and FIG. 11C, the aptamer probes detect IL-8 and VEGF₁₆₅ spiked into serum with very low background, and the signal exhibits a linear dependence upon target protein concentration. The estimated LODs are 3.1 fM (0.026 pg/mL) for IL-8 and 8.9 fM (0.34 pg/mL) for VEGF₁₆₅, demonstrating high sensitivity for both targets. Furthermore, data collected indicated that the high sensitivity of aptamer-based SiMREPS provided detection of endogenous IL-8 in 1:50 diluted (2%) human serum (0.035 pg/mL IL-8, corresponding to 1.75 pg/mL IL-8 in the original serum sample). In contrast, the corresponding sandwich ELISA failed to detect IL-8 in undiluted (100%) human serum, showing that aptamer-based SiMREPS can detect analytes too dilute to assay by conventional methods (FIG. 11D and FIG. 11E).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method for detecting an analyte, said method comprising:
stably binding an analyte to a solid support;
providing an analyte specific aptamer query probe comprising a detectable label; and
recording a time dependent change in a signal intensity of the detectable label.

2. The method of claim 1, wherein the solid support comprises an immobilized capture probe and stably binding the analyte to the solid support comprises stably binding the analyte to the immobilized capture probe.

3. The method of claim 1, wherein the detectable label comprises a fluorescent moiety.

4. The method of claim 1, wherein the solid support is diffusible.

5. The method of claim 1, wherein the analyte comprises a protein.

6. The method of claim 1, wherein the analyte comprises a nucleic acid.

7. The method of claim 1, wherein the analyte comprises a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism.

8. The method of claim 2, wherein the capture probe comprises an antibody or antigen-binding antibody fragment.

9. The method of claim 2, wherein the capture probe comprises a nucleic acid.

10. The method of claim 2, wherein transient association of the query probe with the analyte produces the time-dependent change in the signal intensity of the detectable label.

11. The method of claim 1, further comprising counting a number of changes in the signal intensity of the detectable label.

12. The method of claim 1, further comprising determining a value for Nb+d.

13. The method of claim 1, further comprising determining a value for ton, median.

14. The method of claim 1, further comprising providing a sample comprising the analyte.

15. The method of claim 14, wherein the sample is a biological sample.

16. The method of claim 14, wherein stably binding the analyte to the solid support comprises contacting the sample to the solid support.

17. The method of claim 1, further comprising identifying a candidate aptamer and introducing a number of single nucleotide changes into the conserved target-binding region of the candidate aptamer to produce the aptamer query probe.

18. The method of claim 17, wherein identifying the candidate aptamer comprises using in vitro evolution.

19. The method of claim 17, further comprising truncating the candidate aptamer.

20. The method of claim 14, wherein the analyte is present at a concentration of 1 to 10 fM.

21. The method of claim 14, wherein the analyte is present at a concentration of 0.01 to 1 pg/mL.

22. The method of claim 1, wherein recording the time-dependent change in the signal intensity of the detectable label comprises recording a series of images.

23. The method of claim 22, further comprising producing an intensity fluctuation map by determining an average absolute image-to-image change in intensity at a number of image pixels.

24. The method of claim 22, further comprising generating intensity versus-time data and calculating a kinetic parameter from the intensity-versus-time data.

25. The method of claim 24, further comprising identifying positive detection events using a threshold for the kinetic parameter.

27. A system for detecting an analyte, said system comprising:
a solid support;
an analyte specific aptamer query probe comprising a detectable label;
a detector configured to detect the detectable label;
a memory configured to record time-dependent changes in a signal intensity of the detectable label; and
a processor configured to generate intensity versus-time data from the time-dependent changes in a signal intensity of the detectable label.

28. The system of claim 27, further comprising an analyte.

29. The system of claim 28, wherein the analyte is stably bound to the solid support.

30. The system of claim 27, wherein the solid support comprises a capture probe.

31. The system of claim 27, wherein the detectable label comprise a fluorescent moiety.

32. The system of claim 27, wherein the solid support is diffusible.

33. The system of claim 28, wherein the analyte comprises a protein.

34. The method of claim 28, wherein the analyte comprises a nucleic acid.

35. The system of claim 28, wherein the analyte comprises a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism.

36. The system of claim 30, wherein the capture probe comprises an antibody or antigen-binding antibody fragment.

37. The system of claim 30, wherein the capture probe comprises a nucleic acid.

38. The system of claim 28, wherein transient association of the query probe with the analyte produces the time-dependent change in the signal intensity of the detectable label.

39. The system of claim 27, wherein the processor is further configured to count a number of changes in the signal intensity of the detectable label.

40. The system of claim 27, wherein the processor is further configured to determine a value for N_{b+d}.

41. The system of claim 27, wherein the processor is further configured to determine a value for ι_{on, median}.

42. The system of claim 28, wherein the analyte is present at a concentration of 1 to 10 fM.

43. The system of claim 28, wherein the analyte is present at a concentration of 0.01 to 1 pg/mL.

44. The system of claim 27, wherein the processor is configured to record a series of images.

45. The system of claim 44, wherein the processor is configured to produce an intensity fluctuation map by determining an average absolute image-to-image change in intensity at a number of image pixels.

46. The system of claim 44, wherein the processor is configured to calculate a kinetic parameter from the intensity versus-time data.

47. The system of claim 44, wherein the processor is configured to identify positive detection events using a threshold for the kinetic parameter.

48. Use of an analyte-specific aptamer query probe to characterize, identify, quantify, and/or detect an analyte in a SiMREPS assay method.

49. The use of claim 48, wherein the SIMREPS assay method comprises:
stably binding the analyte to a solid support;
providing the analyte specific aptamer query probe comprising a detectable label; and
recording a time dependent change in a signal intensity of the detectable label.

50. The use of claim 49, wherein the solid support comprises an immobilized capture probe and stably binding the analyte to the solid support comprises stably binding the analyte to the immobilized capture probe.

51. The use of claim 49, wherein the detectable label comprise a fluorescent moiety.

52. The use of claim 49, wherein the solid support is diffusible.

53. The use of claim 48, wherein the analyte comprises a protein.

54. The use of claim 48, wherein the analyte comprises a nucleic acid.

55. The use of claim 48, wherein the analyte comprises a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism.

56. The use of claim 50, wherein the capture probe comprises an antibody or antigen-binding antibody fragment.

57. The use of claim 50, wherein the capture probe comprises a nucleic acid.

58. The use of claim 49, wherein transient association of the query probe with the analyte produces the time dependent change in the signal intensity of the detectable label.

59. The use of claim 49, wherein the SiMREPS assay method further comprises counting a number of changes in the signal intensity of the detectable label.

60. The use of claim 49, wherein SiMREPS assay method further comprises determining a value for Nb+d.

61. The use of claim 49, wherein the SiMREPS assay method further comprises determining a value for ton, median.

62. The use of claim 48, further comprising providing a sample comprising the analyte.

63. The use of claim 62, wherein the sample is a biological sample.

64. The use of claim 62, wherein stably binding the analyte to the solid support comprises contacting the sample to the solid support.

65. The use of claim 49, wherein the SiMREPS assay method further comprises identifying a candidate aptamer and introducing a number of single nucleotide changes into the conserved target binding region of the candidate aptamer to produce the aptamer query probe.

66. The use of claim 65, wherein identifying the candidate aptamer comprises using in vitro evolution.

67. The use of claim 65, further comprising truncating the candidate aptamer.

68. The use of claim 62, wherein the analyte is present at a concentration of 1 to 10 fM.

69. The use of claim 62, wherein the analyte is present at a concentration of 0.01 to 1 pg/mL.

70. The use of claim 49, wherein recording the time-dependent change in the signal intensity of the detectable label comprises recording a series of images.

71. The use of claim 49, wherein the SiMREPS assay method further comprises producing an intensity fluctuation map by determining an average absolute image-to-image change in intensity at a number of image pixels.

72. The use of claim 49, wherein the SiMREPS assay method further comprises generating intensity versus-time data and calculating a kinetic parameter from the intensity versus-time data.

73. The use of claim 49, wherein the SiMREPS assay method further comprises identifying positive detection events using a threshold for the kinetic parameter.
